(19) United States
(12) Patent Application Publication
(10) Pub. No.: US 2010/0143247 A1
(43) Pub. Date: Jun. 10, 2010

(54) DIAGNOSIS OF PROSTATE CANCER

(75) Inventors: Christiane Dorothea Fenske, London (GB); Sabarinath Balachandran Nair, London (GB); Christodoulos Pipinikas, London (GB); Nicholas David Carter, London (GB)

Correspondence Address: FULBRIGHT & JAWORSKI, LLP, 1301 MCKINNEY, SUITE 5100, HOUSTON, TX, 77010-3095, US

(73) Assignee: St. George's Enterprises Limited, Tooting (GB)

(21) Appl. No.: 11/720,103

(22) PCT Filed: Nov. 24, 2005
(86) PCT No.: PCT/GB2005/004494

§ 371 Date: Aug. 13, 2007

(30) Foreign Application Priority Data
Nov. 24, 2004 (GB) 0425873.7
Oct. 21, 2005 (GB) 0521524.9

(51) Int. Cl. A61K 51/00; C12Q 1/68

(52) U.S. Cl. 424/1.61; 435/6

(57) ABSTRACT

The present invention provides a method for determining the presence of prostate cancer in a subject which method comprises determining the level of expression of one or more markers in a blood sample from the subject, wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR.

### CROSS-REFERENCE TO RELATED APPLICATIONS

The present invention claims priority to PCT/GB2005/004494, filed Nov. 24, 2005, Great Britain Application No. 0425873.7, filed Nov. 24, 2004, and Great Britain Application No. 0521524.9, filed Oct. 21, 2005, all of which are incorporated by reference herein in their entirety.

### FIELD OF THE INVENTION

The invention relates to the diagnosis of prostate cancer, in particular to the early diagnosis and staging of prostate cancer.

### BACKGROUND TO THE INVENTION

Prostate cancer (CaP) is increasingly recognised as a major health problem, being the most commonly diagnosed solid cancer and the most common cause of cancer-related deaths in men.

Diagnosis of CaP has been facilitated by the use of two classic criteria, Gleason score and serum PSA. However, despite their prognostic value these criteria have certain limitations. CaP diagnosis using biopsy is difficult and elevated serum PSA levels are not necessarily indicative of CaP, since they have been demonstrated in non-malignant conditions, such as benign prostatic hyperplasia (BPH) and prostatitis. Therefore, the specificity of PSA as a prostate cancer marker is questionable and the subject of ongoing debate. Moreover, due to the fact that CaP patients harbour heterogeneous tumours that vary in progression rates, knowledge about the genes involved in prostate carcinogenesis is still very limited.

Serum PSA measurement may be useful in determining the need for a prostate biopsy, the only alternative diagnostic technique for prostate cancer. A biopsy, carried out under general anaesthetic and taken from the correct area, can be informative, giving information about the presence of cancer, the grade of the tumour and, therefore, how the cancer will develop and eventually spread. However, the test is invasive, painful and, unless the correct area of the tumour is targeted, may not be 100% satisfactory.

Ultrasound and MRI scanning have been used to diagnose the presence of a tumour mass. However, these techniques do not allow the identification of the stage that a tumour or cancer may have reached and cannot distinguish between an enlarged prostate, a benign tumour or a malignant tumour. On the basis of these diagnostic methods, men have been recommended to undergo often unnecessary surgery, which itself carries side effects and reduces quality of life.

There is a need for the development of new, non-invasive and sensitive molecular diagnostic and prognostic CaP tests for the early diagnosis of CaP, the accurate diagnosis of the stage of development of CaP and the monitoring of response to therapy or surgery.

### SUMMARY OF THE INVENTION

The inventors have used relative quantitative RT-PCR (qRT-PCR) to detect marker gene expression in circulating prostate cancer (CaP) cells and have shown that this expression can be used in the diagnosis and monitoring of CaP development and progression. RT-PCR is a powerful technique that is utilized in accordance with the present invention to detect cells that have been shed from the prostate gland into the circulation. The RT-PCR is carried out on mRNA extracted from patients' blood samples. The method is so sensitive that one prostate cell in 100 million blood cells can be detected.

In particular, the inventors' results show highly significant differences in E2F3 gene expression levels in all patient groups (p<0.001): a 39-fold and 14-fold mean increase was found in the localised and metastatic CaP group compared to benign prostatic hyperplasia (BPH) group, respectively. The radical prostatectomy (RP) group showed levels of E2F3 expression similar to those of the localised cancer group, indicating the possible presence of tumour cells in peripheral circulation and suggesting undetected micrometastases. No E2F3 expression was detected in normal male control samples. Correlating E2F3 expression levels in circulating CaP cells with the disease development and progression has diagnostic and clinical implications, suggesting specific therapeutic approaches based on individual gene expression profiles.

The inventors have also used qRT-PCR to detect HIF-1α gene expression in circulating CaP cells and have shown that this expression can be used as an accurate marker in the diagnosis and monitoring of CaP development and progression.

In particular, the inventors found significant differences in the relative HIF-1α expression levels between patients having localized CaP (LocCaP) and the other patient groups (p<0.0001).

In addition, the inventors have used qRT-PCR to detect CAXII gene expressing in circulating CaP cells and have shown that this expression can be used in the diagnosis of CaP and in the monitoring of CaP development and progression. In particular, CAXII expression is up-regulated in the localized CaP group compared to the benign prostatic hyperplasia group and down-regulated in the metastatic CaP group compared to the localized CaP group.

The inventors have also identified other markers of prostate cancer that may be detected on circulating CaP cells and used either alone or in combination with other prostate cancer markers in methods of prostate cancer diagnosis and/or staging or prostate cancer using qRT-PCR analysis of marker expression in cells present in bodily fluids, such as cells circulating in the blood. These additional markers include c-met, pRB, EZH2, e-cad, CAIX, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR. The sensitive non-invasive qRT-PCR techniques provided by the invention may utilise any one or more of the above mentioned markers.

The inventors have additionally demonstrated using RT-PCT that CaP is associated with alternative splice variants of certain markers including E2F3, e-cad and CAIX.

Accordingly, the present invention provides:
a method for determining the presence of prostate cancer in a subject which method comprises determining the level of expression of one or more markers in a blood sample from the subject;
a method for determining the stage of prostate cancer in a subject, which method comprises determining the level of expression of one or more markers in a blood sample from the subject;
a method for monitoring the response of a subject to prostate cancer treatment, which method comprises determining the level of expression of one or more markers in a blood sample from the subject; and
a method for determining the aggressiveness of prostate cancer in a subject, which method comprises monitoring the level of expression of one or more markers in a blood sample from the subject.

In each of the above methods, the markers preferably comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR. The methods may each further comprise determining the presence or absence of one or more alternative splice variant of one or more marker.

The invention also provides:
a test kit suitable for use in a method for determining the presence of prostate cancer in a subject, which test kit comprises means for determining the level of expression of one or more markers in a blood sample from the subject, wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR;
use of an agent in the manufacture of a medicament for use in the treatment of prostate cancer in a subject, wherein the subject has been identified as having prostate cancer according to a method of the invention; and
a method for the treatment of prostate cancer in a subject, which method comprises:
   (a) determining whether the subject has prostate cancer by use of a method according to the invention; and
   (b) administering to a subject identified in (a) as having prostate cancer, a therapeutically effective amount of an agent used in the treatment of prostate cancer.

### BRIEF DESCRIPTION OF THE DRAWINGS

FIG. 1 shows relative quantitative E2F3 expression levels in four patient groups. Expression of E2F3 was massively up-regulated in the LocCaP patient group indicating a possible diagnostic and prognostic implication for the early diagnosis and accurate staging of CaP.

FIG. 2 shows the probability that the patient is predicted to belong to each group versus their relative levels of E2F3 expression (predicted based on multinomial regression model including E2F3/GAPDH ratio as the explanatory variable).

FIG. 3 shows the probability that the patient is predicted to belong to each group versus their relative levels of E2F3 expression (predicted based on multinomial regression model including E2F3/GAPDH ratio and Gleason score as the explanatory variables).

FIG. 4 shows the discrimination of predicted probabilities classified as (a) BHP (b) LocCaP and (c) MetCaP split according to true diagnosis for the 82 patients taking part in the study (predictions based on multinomial regression including E2F3/GAPDH ratio as the explanatory variable).

FIG. 5 shows the results of quantitative RT-PCR of HIF-1α RNA using LightCycler™ and SYBR Green I. Plot of fluorescence signal during amplification. Serial dilutions of purified HIF-1α PCR product were prepared and used as external standards for data normalisation (A) and graph of crossing points (Cₚ—cycle number) plotted against the log of copy numbers (concentration) to obtain a standard (calibration) curve (B). Melting curve analysis demonstrated the presence of a narrow peak formed at 82° C. (C) and 1% agarose gel electrophoresis showing a single band at the expected size of 418 bp (D).

FIG. 6 shows a logarithmic plot of relative quantitative HIF-1α expression levels in the different patient groups (A) and pair-wise comparisons of HIF-1α expression using ANOVA (B).

### DETAILED DESCRIPTION OF THE INVENTION

According to the invention, there is provided a method for the identification of a subject in which CaP is present. The invention also provides a method for distinguishing between patients with no evidence of malignancy (NEOM), localised prostate cancer (LocCaP) and/or metastatic prostate cancer (MetCaP), a method for determining the aggressiveness of CaP and a method for monitoring CaP.

The diagnostic tests provided by the present invention use patient blood samples. The methods detect circulating normal prostate cells, cancer cells and prostate cancer cells using quantitative analysis of expression levels of candidate markers in the blood or other bodily fluid. The markers may be identified by any suitable technique such as by tissue analysis or SELDI-ToF analysis using protein chips. Any marker which is up-regulated or down-regulated in CaP or at different stages of CaP may be monitored using the non-invasive molecular technique of the invention. The test may involve analysis of a single marker, but analysis of a combination of two or more markers is preferred.

A method of the invention may comprise monitoring the level of expression of at least one of E2F3, HIF-1α, CAXII, CAIX, EZH2, PIM-1, c-met, e-cad, Jagged, hepsin, pRB, RECK, Clusterin, MMP9, MTSP-1, MMP24, MMP15, IGFBP-2, E2F4, IGFBP-3, caveolin, EF-1A, Kallikrein 2, Kallikrein 3 and PSGR.

The method can be used to identify a subject in which CaP is at a very early stage. In addition, the method may be used to discriminate between different stages of CaP, i.e. to stage CaP. The method may also be used to monitor the effectiveness of a CaP therapy (either when the therapy is taking place or after the therapy has ceased). Thus, the method may be used to identify a subject suffering from micrometastases when all cancer tissue has apparently been excised by surgery.

The subject may be asymptomatic for CaP when the method of the invention is carried out. Preferably, the subject exhibits one or more symptom that is potentially due to prostate problems. Preferably, the subject is a mammal, for example a human.

### Sample

The method of the present invention is a non-invasive method that can be carried out without requiring a biopsy. The present method detects expression of prostate cancer markers on prostate cells present in a body fluid, such as whole blood. Typically, the body fluid used in the method of the invention is blood. Other body fluids that may be used include urine and cerebrospinal fluid. The method of the invention may be carried out in vivo, although more usually it is convenient to carry out the method in vitro or ex vivo on a sample derived from the subject.

The sample may be processed in order that the method may be carried out. For example, nucleic acid extraction may be carried out. In particular, RNA, such as total RNA or mRNA, may be isolated. RNA extracted from a sample may subsequently be converted into cDNA. The polynucleotide in the sample may be copied (or amplified), for example by using a PCR-based technique.

The sample may be processed in a test of the invention immediately after being obtained from the patient. Alternatively, the sample may be stored under conditions under which mRNA and/or protein remains stable. For example, the sample may be kept on ice, frozen or stored in a blood tube (Bioanalytix) or other container that keeps mRNA stable at ambient temperature (i.e. at about 20° C.).

### Determination of Marker Expression

Determination of the presence of absence of expression of the marker genes may be carried out at the RNA level, for example by determining the presence or absence of RNA, in particular mRNA, or at the protein level, for example by determining the presence or absence of the marker gene product. For example, determination of the presence of absence of expression of the marker genes may be carried out at the nucleotide, for example RNA level by RNA blotting or reverse transcriptase-PCR(RT-PCR), and/or at the protein (polypeptide) level by use of an antibody.

Determining the presence or absence of marker gene expression may involve determining the amount of expression of the marker gene in the subject, i.e. the level of expression of the marker gene in a body fluid of the subject may be determined. This may be carried out at the RNA and/or the protein level. The level of expression of the marker gene may be determined absolutely or relatively, for example in relation to an internal control chosen because the level of expression of such a gene remains more or less constant, for example substantially constant, in cancerous and non-cancerous cells. The analysis of marker gene expression in a subject may thus be quantitative as well as qualitative.

In one preferred embodiment, quantitative real time PCR (qRT-PCR) analysis is used to determine the expression levels of the marker. qRT-PCR is an extremely sensitive technique for measuring gene expression levels and hence can be used to detect marker expression on tumour cells in peripheral circulation.

In one preferred embodiment, quantitative real time PCR (qRT-PCR) analysis is used to determine the expression levels of the marker. qRT-PCR is an extremely sensitive technique for measuring gene expression levels and hence can be used to detect marker expression on tumour cells in peripheral circulation.

qRT-PCR uses primers for an internal control that are multiplexed in the same RT-PCR reaction with the gene specific (i.e. marker gene specific) primers. The internal control and gene-specific primers must be compatible, i.e. they must not produce additional bands or hybridize to each other. The expression of the internal control should be constant across all samples being analyzed. Then the signal from the internal control can be used to normalize sample data to account for tube-to-tube differences caused by variable RNA quality or RT efficiency, inaccurate quantitation or pipetting.

Internal controls suitable for use in the invention include genes encoding enzymes of the glycolytic pathway, such as glyceraldehyde-3-phosphate dehydrogenase (GAPDH), α-actin and β-actin. For qRT-PCR, the PCR reaction is typically terminated when the products from both the internal control and the marker gene product are detectable and are being amplified within exponential phase. Because internal control RNAs are typically constitutively expressed housekeeping genes of high abundance, their amplification surpasses exponential phase with very few PCR cycles. Detecting a rare message while staying in exponential range with an abundant message can be achieved several ways: 1) by increasing the sensitivity of product detection, 2) by decreasing the amount of input template in the RT or PCR reactions and/or 3) by decreasing the number of PCR cycles.

In one embodiment of the present invention, determination of the presence or absence of expression of marker gene expression may involve determining the presence or absence of one or more alternative splice variant of one or more marker. In this embodiment, the presence or absence of an alternative splice variant is typically detected by RT-PCR using primers which bind specifically to the nucleotide sequences which flank the region or regions where alternative splicing occurs. The presence of alternative splice variants may also be detected at the protein level.

The oligonucleotide primers used in the present invention for amplification of the marker gene and the internal control are capable of acting as an initiation point for synthesis when placed under conditions which induce synthesis of a primer extension product complementary to a nucleic acid strand. The conditions can include the presence of nucleotides and an inducing agent such as a DNA polymerase at a suitable temperature and pH.

Sensitivity and specificity of the oligonucleotide primers are determined by the primer length and uniqueness of sequence within a given sample of template DNA. Primers which are too short, for example less than about 10 mers, may show non-specific binding to a wide variety of sequences in the genomic DNA and are not preferred for use in this invention.

Thus a primer used in the invention will be sufficiently long to prime the synthesis of extension products in the presence of the agent for polymerization and typically will contain from about 10 to about 50 nucleotides. Shorter primer molecules generally require cooler temperature to form sufficiently stable hybrid complexes with the template. Preferably, a primer used in the methods of the invention may be from about 15 to about 35 nucleotides in length, for example from about 18 to about 30 nucleotides in length. The melting temperature (Tₘ) of a primer used in the invention will typically be from about 50° C. to about 70° C. The primers do not need to be of the same length or have the same melting temperature.

A primer suitable for use in the methods of the invention may occur naturally, for example as in a purified restriction digest, or may be produced synthetically or recombinantly. Methods for the preparation of synthetic or recombinant oligonucleotides are well known to those skilled in the art. Suitable methods for the preparation of synthetic oligonucleotides include preparation using the triester method or phosphoramidite chemistry. Suitable methods for the preparation of recombinant oligonucleotides include preparation by enzymatically directed copying of a DNA or RNA template.

A primer suitable for use in the invention may be chemically modified. For example, phosphorothioate primers may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-β-methylribonucleotide methylphosphonates.

Alternatively mixed backbone primers (MBOs) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides.

In general, suitable PCR primers will comprise sequences entirely complementary to the corresponding sequence to be amplified. However, if required, one or more, for example up to about 3, up to about 5 or up to about 8 mismatches may be introduced, to introduce a convenient restriction enzyme site for example, provided that such mismatches do not unduly affect the ability of the primer to hybridize to its target sequence. Suitable primers may carry one or more labels to facilitate detection.

Any part of each of the marker genes may be used as a target for a PCR primer, although typically a region is used which does not share substantial homology with other genes. The PCR primers used may be designed so that all or part of each of the marker mRNAs is amplified. The same principles apply to the design of primers for the amplification of internal control mRNAs.

Examples of suitable primers for qRT-PCR are shown in Table 3. Examples of suitable primers for detecting alternative splice variants are shown in Table 9.

Relative quantitative RT-PCR may be carried out for each marker gene being used and an internal control gene using, for example, a LightCycler™ (Roche) and SYBR Green I according to manufacturer's protocol.

Levels of marker mRNA and internal control mRNA may be calculated using the construction of calibration curves using purified marker PCR product and an internal control plasmid, respectively. Relative quantification may be calculated as a ratio of the amount of target molecule marker gene divided by the amount of internal control, e.g. marker/internal control. Points from the marker gene and internal control standard curves may be included in each subject run, to enable accurate calculation of relative quantification.

Melting curve analysis may be carried out following quantification to confirm the specificity of the qRT-PCR reaction and to distinguish between specific and non-specific marker products and primer dimers. In addition, marker qRT-PCR products may be electrophoresed, for example on 1% agarose gels to confirm melting curve analysis results.

### Diagnosis of CaP

The method for determining the presence of CaP in a subject typically comprises determining the level of expression of one or more markers in a body fluid sample, typically a blood sample, from the subject, typically by RT-PCR.

A method of the invention may comprise monitoring the level of expression of at least one of E2F3, HIF-1α, CAXII, CAIX, EZH2, PIM-1, c-met, e-cad, Jagged, hepsin, pRB, RECK, Clusterin, MMP9, MTSP-1, MMP24, MMP15, IGFBP-2, E2F4, IGFBP-3, caveolin, EF-1A, Kallikrein 2, Kallikrein 3 and PSGR.

In one embodiment, the method comprises determining the presence or absence, preferably the level, of E2F3 gene expression. The E2F3 gene is a member of the E2F family of transcription factors.

One or more of the above specified markers, for example two, three, four, five, six or all of them, may be used in combination. Preferred markers for diagnosing CaP in patient, or for determining whether a patient does not have CaP, include RECK, Clusterin, MMP9, E2F3, HIF-1α, MTSP1 and e-cad. Preferred combinations for diagnosing CaP, or for determining whether a patient does not have CaP include: RECK and Clusterin; Clusterin and MTSP1; RECK, Clusterin and MTSP1; and RECK and MTSP1.

The presence or absence of the expression, and preferably the level of expression of further markers in the blood sample of a subject may be determined in addition to the presence or absence of expression, preferably the level of expression, of the above mentioned markers such as E2F3, HIF-1α and/or CAXII gene expression. For example, the presence or absence of expression of from one, two, three, four, five or more genes up to about 10, about 20, about 50, about 100 or about 500 or more genes may be determined. Thus, the presence or absence of the expression of each of a panel of genes, of which at least one may be one of the markers specified herein, such as the E2F3 gene, the HIF-1α gene and/or the CAXII gene, may be determined. The level of expression of the each of the marker genes may be determined. This may give a more accurate indication of the stage of CaP in a subject. In addition, treatment may then be tailored to the particular expression profile of a subject.

Additional genes, the presence or absence of the expression of which or the level of expression of which, may be determined in the method of the invention, include one or more of AMACR, PSA and FAS in the body fluid sample may be determined to further enhance the diagnosis, staging test, relapse monitoring test or aggressiveness test. Typically, the level of expression of each gene that is used will be determined.

The expression levels of each of the marker genes being utilized may be determined simultaneously, for example in a multiplex qPCR reaction, or separately in individual qPCR reactions.

The nucleotide sequence of the marker genes mentioned herein are set out in GenBank under the accession numbers indicated in the Table below.

| Marker Gene | Accession Number |
|---|---|
| RECK | NM_021111 |
| HIF-1α | NM_001530 and NM_181054 |
| Pim-1 | NM_002648 |
| MMP9 | NM_004994 |
| MMP15 | NM_002428 |
| CAXII | NM_206925 and NM_001218 |
| CAIX | NM_001216 |
| ICFBP-2 | NM_000597 |
| IGFBP-3 | NM_000598 and NM_001013398 |
| FAS | NM_004104 |
| EF-1A | NM_001402 |
| MTSP-1 | NM_021978 |
| E2F3 | NM_001949 |
| E2F4 | NM_001950 |
| AMACR | NM_014324 and NM_203382 |
| EZH2 | NM_152998 and NM_004456 |
| Caveolin | NM_001753 |
| E-cad | NM_004360 |
| Kallikrein-2 | NM_005551 |
| Kallikrein-3 | NM_001648 |
| MMP2 | NM_004530 |
| MMP24 | NM_006690 |
| Clusterin | NM_001831 |
| Hepsin | NM_002151 |
| c-met | NM_000245 |
| PSGR | AF369708 |
| pRB | NM_000321 |

### Monitoring Stage of CaP

The expression levels of each of the CaP markers described herein may be used to monitor the stage of CaP development in patients known to have the disease. The method of diagnosis may give an indication of the stage of disease in a previously undiagnosed patient. For example the diagnosis may indicate that the patient has early stage CaP, such as localised CaP. The different marker genes are differentially up-regulated and/or down-regulated at different stages of tumour development. Therefore, an increased level of one marker may be accompanied by a decreased level of a different marker.

Expression levels of different markers are gradually up-regulated between the various stages of CaP and so there is no sharp cut off point between stages. The markers that play a part in the early stages of the CaP may be different to those involved at the later stages. For example, the hypoxia which is associated with cancer formation in the early stages of the disease results in the up-regulation of hypoxia-inducible markers such as HIF-1α and carbonic anhydrases. HIF-1α mediates activation of genes involved in cell survival and apoptosis. These normal cellular responses also give tumour cells a survival advantage. However, once the tumour is established, other mechanisms take over during tumour development. At this stage, expression of other genes and markers, such as caveolin or MMP9, may be induced or up-regulated.

Therefore, a diagnostic test that is able to distinguish between all stages of prostate cancer development typically involves several markers with each marker being specifically regulated and sensitive to the different stages of CaP. A combination of markers may be used to increase the accuracy of diagnosis of any one stage. For example, specific markers with low sensitivity may be combined with sensitive markers with low specificity.

In one embodiment of the invention, the expression levels of one or more CaP markers in the blood, or other bodily fluid, may be used to determine whether a patient has no evidence of malignancy (NEOM) or localised CaP (LocCaP). Preferred markers for distinguishing NEOM and LocCaP include RECK, Clusterin, HIF-1α, E2F3, MMP9 and E2F4.

In another embodiment of the invention, expression levels of one or more CaP markers in the blood, or other bodily fluid, may be used to distinguish LocCaP from MetCaP. Preferred markers for distinguishing LocCaP and MetCaP include RECK, Clusterin, HIF-1α, IGFBP-3, E2F3, caveolin, MMP9, PIM-1 and MTSP1.

For example, the amount of E2F3 gene expression in a body fluid of a subject may be used to discriminate between the different stages in the development of CaP. Thus, low levels of E2F3 gene expression may be indicative of benign hyperplasia (BPH or NEOM), whereas high levels of E2F3 gene expression may be indicative of malignant CaP (MetCaP). Thus, the level of E2F3 gene expression may be used to discriminate between a subject suffering from a benign CaP and a subject suffering between a malignant CaP.

Also, the amount of E2F3 gene expression in a sample may be used to discriminate between localised invasive CaP and metastatic CaP. The discrimination between these two types of cancer may be further enhanced if the level of E2F3 gene expression is considered together with expression of other markers and/or with other diagnostic indicators of CaP, for example the Gleason score and/or levels of serum PSA.

In one embodiment of the invention, the level of CAXII gene expression in a body fluid sample from a subject may be used to determine the stage of CaP development in the subject, either where the subject is known to have CaP or as part of a CaP diagnostic test. In particular, high levels of CAXII gene expression may be indicative of localized CaP (LocCaP), whereas low levels of CAXII gene expression may be indicative of malignant CaP (MetCaP) or benign prostatic hyperplasia (BPH). Again, the discrimination between the types of cancer may be further enhanced if the level of CAXII gene expression is considered together with expression of other markers and/or with other diagnostic indicators of CaP such as the Gleason score and/or levels of serum PSA. In particular, consideration of CAXII levels in combination with detection of other markers may be useful to distinguish benign prostatic hyperplasia from metastatic CaP.

In a further embodiment, levels of HIF-1α expression in the body fluid may be detected to discriminate between different stages in the development of CaP. In particular, high levels of HIF-1α may be indicative of localized CaP, whilst low levels of HIF-1α may indicate that the patient has benign prostatic hyperplasia or that the cancer has become metastatic. Again, use of HIF-1α as a marker in combination with one or other markers may be useful to distinguish between the different stages of CaP, for example between benign prostatic hyperplasia and metastatic CaP.

### Prognostic Value of Markers

In addition to changes in their expression levels, some markers, such as E2F3 and CAIX, have prognostic value in determining the aggressiveness of disease development. The inclusion of one or more such marker in a panel of markers used in the diagnostic test of the invention would not only contribute to accurate diagnosis of the stage of the disease, but also be able to indicate speed of potential disease progression. The ability to predict the speed of disease progression will enable an appropriate choice of therapy to be made.

E2F3 is a suitable marker for determining the likely aggressiveness of CaP as it has previously been reported to be associated with aggressive forms of prostate cancer (Foster et al., Oncogene, 2004, 23(35):5871-5879).

CAIX may be used to predict the aggressiveness of CaP in a patient. CAIX is hypoxia induced and is up-regulated early on in the development of CaP. A potential additional splice form is expressed in an androgen-independent bone cell line (PC3) which is non-responsive to therapy, but not in androgen-dependent lymph node cell line (LnCaP). The alternatively spliced form is also present in all patient samples. This suggests that CAIX is up-regulated in aggressive tumours and that the alternatively spliced form is an early diagnostic and prognostic tool for this.

### Application of Test at the Metastatic Stage

Current methods for diagnosing CaP at the metastatic stage include the measurement of serum PSA levels, with levels>10 μg/l being indicative of metastatic cancer. However, some patients with localised cancer and those with no evidence of malignancy may have higher levels and some patients with metastatic cancer may have lower levels. Therefore, this method cannot be used by itself for accurate diagnosis.

Tumour biopsies also cannot be used to accurately diagnose the metastatic stage of CaP because histology does not show whether the tumour has become metastatic or whether it remains localised. Bone scans may be used to identify any secondary tumours. However, their visualisation depends on whether the tumours are large enough and established enough to be detected. Therefore, early stages of metastatic disease may not be identified. The identification of metastatic CaP at this early stage of development is essential so that appropriate effective therapy may be sought

The qRT-PCR diagnostic test of the present invention detects cells circulating in peripheral blood. Markers which are specifically up-regulated at the metastatic stage may be selected for monitoring to determine whether a patient has metastatic CaP. For example, MMP9 is up-regulated in LocCaP compared to BPH/NEOM and is significantly further up-regulated in MetCaP giving clear indication of metastatic spread. Therefore, any increase in the level of MMP9 or other markers which are up-regulated or down-regulated in MetCaP would indicate a risk of metastatic spread. The test of the present invention allows any increase in expression levels of the marker, or increase in circulating cell numbers to be detected. Monitoring changes in expression of the same markers may be carried out to determine the effectiveness of therapy. Effective treatment would result in a reduction in cell numbers and reduced marker expression.

RECK, Clusterin, HIF-1α, IGFBP-3, E2F4, caveolin, PIM-1 and MTSP1 are other preferred markers that may be used to determine whether CaP is metastatic.

### Monitoring for Possible Relapse Post Surgery/Effectiveness of CaP Therapy

In another embodiment of the invention, expression levels of the markers mentioned herein in a body fluid sample from a subject may be used to monitor a patient who is being treated for CaP or who has been treated for CaP.

For example, in post-operative CaP patients, typically patients who have had a radical prostatectomy (RP), analysis of marker expression in the blood may be used to monitor the likely re-occurrence of CaP. If, following surgery, marker levels continue to increase or show no signs of decreasing, this may indicate either residual disease or previously undetected metastases. This may be illustrated with reference to HIF-1α. In post-operatively obtained blood samples from patients who have undergone radical prostatectomy (RP), patients with positive surgical margins indicative of residual disease have significantly higher HIF-1αlevels in the blood than patients with negative surgical margins who do not show signs of residual disease. Hence, HIF-1α may be used alone or in combination with one or more other markers to monitor disease relapse.

In patients undergoing hormone treatment or radiotherapy for LocCaP, an increase in expression levels of some markers or a decrease in expression levels of other markers indicate a continuing risk of the cancer developing to the metastatic stage. This would dictate alternative or more aggressive therapy. Conversely, if the therapy is successful, marker expression levels would become similar to levels of typical of BPH/NEOM.

MTSP1 and E2F3 markers are preferred in this embodiment and RECK, Clusterin and MMP9 are more preferred. These markers all show highly significant differences between NEOM/BPH and MetCaP patient groups.

### Analysis of Expression Levels

For any particular combination of marker and internal control, statistical analysis may be carried out such that a probability can be generated of a given level of marker gene expression (as determined by qPCR) being indicative of, a particular stage of prostate cancer, for example benign versus malignant or localised invasive versus metastatic.

For example, results from different patient groups may be analysed using ANOVA and multiple comparisons for all pair-wise contrasts of the relative marker expression levels between patient groups. An alternative non-parametric method (Kruskal-Wallis rank sum test) may also be used to determine differences in relative marker expression between patient groups. In addition, data may be further analysed using a multinomial model.

The data may be analysed using a statistical technique that analyses two or more variables at a time (multivariate analysis), such as: discriminant analysis, factor analysis, cluster analysis, logistic regression, ANOVA or principal component.

Discriminant analysis, for example, is a technique for classifying a set of observations into predefined classes. The aim is to determine the class of an observation based on a set of variables known as predictors or input variables. The model is built based on a set of observations for which the classes are known. For example, in the test for diagnosis of prostate cancer and differential diagnosis of the different stages of the disease, discriminant analysis may be used to identify the features which are responsible for splitting a set of observations into two or more groups, such as cancer and non-cancer patients. Information about individual cases is obtained from a number of variables. It is reasonable to ask if these variables can be used to define groups and/or predict the group to which an individual belongs. Discriminant Analysis works by creating a new variable that is a combination of the original variables. This is done in such a way that the differences between the predefined groups are maximized.

Receiver Operator Characteristic/Area Under Curve (ROC/AUC) analysis is another technique that may be used to analyse data obtained using a test of the invention (Metz, C. E. (1978), Basic principles of ROC analysis, Semin Nucl Med. 8(4):283-98). ROC/AUC analysis enables diagnostic “accuracy”, “sensitivity” and “specificity” of a diagnostic test to be measured. These measures and the related indices, “true positive fraction” and “false positive fraction” depend on the arbitrary selection of a decision threshold. The receiver operating characteristic (ROC) curve is shown to be a simple yet complete empirical description of this decision threshold effect, indicating all possible combinations of the relative frequencies of the various kinds of correct and incorrect decisions.

The accuracy of the test depends on how well the test separates the group being tested into, for example, those with and without CaP. Accuracy is measured by the area under the ROC curve (AUC). An area of 1.0 indicates that the test has a maximum discriminatory power; an area of 0.5 represents no discrimination. A rough guide for classifying the accuracy of a diagnostic test is as follows:
0.90-1=Maximum discriminatory power
0.80-0.90=Good discrimination
0.70-0.80=Fair discrimination
0.60-0.70=Poor discrimination
0.50-0.60=Fail no discriminatory power

The method by which it is determined whether the levels of the CaP markers are indicative of CaP or non-cancer, whether the levels of the CaP markers are indicative of a particular stage of CaP such as LocCap or MetCap, or by which the aggressiveness of CaP in a patient is predicted may be implemented using a computer. The computer may be physically separate from or may be coupled to the reader used to generate expression data, for example to the LightCycler™.

Supervised machine learning classification methods may be used to discriminate non-cancer, CaP and/or the various stages and/or the aggressiveness of CaP using expression data obtained by qRT-PCR. The machine learning classifier is first trained using training expression data from patients whose condition is known and training control data from control subjects.

Suitable machine learning classifiers include the single layer perceptron (SLP), the multi-perceptron (MLP), decision trees and support vectors machines. Preferably the classifier in a support vector machine such as a Gaussian kernel support vector machine. Other suitable bioinformatics models may also be used such as purely biostatistical algorithms, genetic cluster algorithms and decision classification trees.

### CaP Symptoms and Diagnosis

Markers of CaP may be up-regulated in patients with localised cancer (compared with those with NEOM or BPH), and further up-regulated in patients with metastatic disease (see Marker 1 in the Table below). Example of such markers include caveolin and MMP9. Determining the level of expression of one or more such markers enables accurate diagnosis of all stages of disease development.

Other markers of CaP are up-regulated in patients with localised cancer, but are then down-regulated in patients with metastatic disease (see Marker 2 in the Table below). An example of such a marker is e-cad. In some cases, levels of marker expression in patients with no evidence of malignancy and in patients with metastatic disease may be quite similar. In such cases, other factors may be taken into consideration in order to distinguish between the two forms of the disease. For example, the markedly different symptoms and serum PSA levels between NEOM patients and MetCaP patients may be monitored in addition to the CaP markers on circulating CaP cells.

| | Normal males | BPH/NEOM | LocCap | MetCap |
|---|---|---|---|---|
| Marker 1 | 0 | 0-+ | +++ | ++++ |

= accurate diagnosis of all stages

| | | | | |
|---|---|---|---|---|
| Marker 2 | 0 | 0-+ | +++ | + |

= confusion between BPH/NEOM and MetCap

| | | | | |
|---|---|---|---|---|
| Serum PSA | >0.1 | 0.1-4 | 4-10 | >10 |

Inclusion of serum PSA in results aids accurate differential diagnosis

Levels of serum PSA below 4 are traditionally taken as being indicative of NEOM, and levels above 4 are indicative of localised cancer. However, due to an area of overlap, serum PSA levels are unable to accurately distinguish between benign and localised cancer. The highly significant up-regulation of the markers included in this study in localised cancer patients may be used in accurate differential diagnosis of these disease stages. Down-regulation of these markers at the metastatic stage, however, are accompanied by marked elevation of serum PSA (levels above 10 indicate metastatic disease). Therefore, it is possible to accurately diagnose a patient with no evidence of malignancy (serum PSA<4) from one with metastatic disease (>10), despite the fact that expression of some markers may be quite similar.

Some of the markers of CaP also serve as markers of other types of cancer. Therefore, a diagnosis of CaP is typically made where the patient exhibits physical symptoms characteristic of CaP. Thus, symptoms may be used to distinguish CaP from other cancers or to help distinguish different stages of CaP.

Patients with BPH/NEOM present with increased frequency of urination (nocturia), delay or difficulty in initiating urination, dribbling, sensation of incomplete bladder emptying and reduced urine stream. Obstructive symptoms may also lead to hydronephrotic changes and even renal failure if untreated. Development of these symptoms are usually prolonged, typically from months to years.

Patients with metastatic disease often have a more rapid onset of symptoms, typically within a few months. In addition to the above symptoms, the patient may demonstrate haematuria, possibly with accompanying anaemia due to blood loss. This along with cancer cachexia may lead to tiredness and lethargy. Metastases form primarily in the bone with the hip, pelvis, spine (lumbar and thoracic regions) and rib cage being the most common sites. This results in pain that does not resolve with rest and which does not respond to the usual analgesics. The metastases in the bone may lead to spontaneous fractures, which may lead to neurological deficits. Lumbar fractures may lead to sensorimotor deficits in the lower limbs and may also result in incontinence. All these symptoms will not be present in a patient presenting with BPH or NEOM.

Additional examinations which may further clarify diagnosis include:
bone scans to identify the presence of metastases;
analysis of urine electrolytes to demonstrate sudden increase in levels of creatinine in metastatic patients;
monitoring levels of alkaline phosphatase levels in blood samples with elevated levels indicating the presence of bone metastases; and
rectal examination to demonstrate a prostate gland that is smooth in BPH or hard and irregular when malignant.

In order to achieve optimal results concerning the inclusion of other covariates, the AIC criterion may be taken into account. Gleason score and age may additionally or alternatively be included as covariates to determine their effect on AIC score and hence effect on model suitability.

Control assays may be carried out, for example using a sample derived from a non-cancer subject or a sample derived from a subject known to have CaP.

### Test Kits

The invention also provides a test kit for use in a method of the invention. The test kit maybe for diagnosing CaP, determining the stage of CaP, determining the aggressiveness of CaP, monitoring potential relapse in post-operative patients or monitoring the effectiveness of therapy in patients. A test kit of the invention therefore comprises means for determining the presence or absence, or level of expression of, one or more markers in a body fluid sample from a subject, wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR. The test kit may further comprise means for determining the level of expression of one or more of AMACR, PSA and FAS in a body fluid sample from a subject.

Means for determining the presence or absence of marker gene expression in a body fluid of a subject may comprise, for example, means for determining the level of expression of the marker gene in body fluid of a subject, in particular means for determining the level of expression of the marker gene in body fluid of a subject using relative quantitative PCR. Means for determining the presence or absence of marker gene expression may comprise means for determining the presence or absence of an alternatively spliced form of one or more marker.

Any suitable means for determining the determining the presence or absence of marker gene expression in a body fluid of a subject may be included in a test kit of the invention. Typically, the means will comprise two oligonucleotides (primers) which can be used to amplify a marker. Typically a primer pair will be included for each marker of interest. A test kit of the invention may optionally comprise appropriate buffer(s), enzymes, for example a thermostable polymerase such as Taq polymerase and/or control polynucleotides. A kit of the invention may also comprise appropriate packaging and instructions for use in a method for determining the susceptibility of a subject to stroke. A test kit of the invention may also comprise an agent which is used in the treatment of CaP.

The kit may comprise a container for the storage and/or transport of the sample, preferably blood, or a processed form of a sample, such as RNA extracted from the sample. The container may be one capable of keeping mRNA stable at ambient temperature (i.e. at about 20° C.) for from about 1 to about 10 days, and preferably for at least about 4 days. For example, the container may be a blood tube (Bioanalytix). The advantage of using such a container is that patients would not need to travel to the site of testing, such as a hospital.

### CaP Treatment

The invention allows the identification of a subject having CaP at a very early stage of development of the cancer. The CaP can thus be treated at an early stage of its development. A patient identified as suffering from a CaP may be treated for CaP. Any suitable treatment or therapy which is known for the treatment of CaP may be used.

Watchful waiting may be appropriate. This involves closely monitoring a patient's condition without giving any treatment until symptoms appear or change. This is usually used in older men with other medical problems and early-stage disease.

Patients in good health who are younger than 70 years old are usually offered surgery as treatment for CaP. The following types of surgery may be used to treat a patient identified according to the invention:
pelvic lymphadenectomy: this is a surgical procedure to remove the lymph nodes in the pelvis. A pathologist views the tissue under a microscope to look for cancer cells. If the lymph nodes contain cancer, the doctor will not remove the prostate and may recommend other treatment;
radical prostatectomy: this is a surgical procedure to remove the prostate, surrounding tissue, and nearby lymph nodes. There are 2 types of radical prostatectomy: retropubic prostatectomy, a surgical procedure to remove the prostate through an incision (cut) in the abdominal wall; and perineal prostatectomy, a surgical procedure to remove the prostate through an incision (cut) made in the perineum (area between the scrotum and anus). Removal of nearby lymph nodes may be done at the same time as either type of radial prostatectomy;
transurethral resection of the prostate (TURP): a surgical procedure to remove tissue from the prostate using a cystoscope (a thin, lighted tube) inserted through the urethra. This procedure is sometimes done to relieve symptoms caused by a tumour before other cancer treatment is given. Transurethral resection of the prostate may also be done in men who cannot have a radical prostatectomy because of age or illness.

Impotence and leakage of urine from the bladder or stool from the rectum may occur in men treated with surgery. In some cases, a technique known as nerve-sparing surgery may be used. This type of surgery may save the nerves that control erection. However, men with large tumours or tumours that are very close to the nerves may not be able to have this surgery.

Radiation therapy may be used in which high-energy x-rays or other types of radiation are used. The radiation therapy may be external radiation therapy or internal radiation therapy. Internal radiation therapy uses a radioactive substance sealed in needles, seeds, wires, or catheters that are placed directly into or near the cancer. The way the radiation therapy is administered will depend on the type and stage of the cancer being treated.

Hormone therapy may include the following: luteinizing hormone-releasing hormone agonists such as leuprolide, goserelin, and buserelin; antiandrogens such as flutamide and bicalutamide; drugs that can prevent the adrenal glands from making androgens including ketoconazole and aminoglutethimide. Orchiectomy may also be used to decrease hormone production.

Cryosurgery may be used in which CaP cells are frozen and thereby destroyed.

Treatment of metastatic prostate cancer (MetCaP) involves local treatment of the prostate and the treatment of secondary tumours.

Local treatment for the prostate typically involves hormone therapy as the first line. For example luteinising hormone-releasing hormone (LHRH) agonists and/or anti-androgen therapy (Bicalutamide or Cyproterone acetate) may be used. Once patients fail hormone therapy, the treatment is advanced to include various modalities of chemotherapy, such as Corticosteroids, Mitoxantrone, Docetaxel, Suramin and/or Estramustines. Radiotherapy may be given to the prostate in localized cancer if there is persistent haematuria or secondary effects such as hydronephrosis (dilation of the kidney/ureter) leading to renal failure.

Secondaries are usually present in bone. Treatment of secondaries typically involves local radiotherapy or radiopharmaceutical agents such as Strontium-89. Patients may be treated for pain or neurological compromise. Bisphosphonates can be used for treatment related osteoporosis and to reduce incidence of skeletal related events.

Other supportive measures include treatment of anemia and bleeding, management of disseminated intravascular coagulation (if it occurs), opioids for pain control, ureteral stenting or diversion for ureteral obstruction.

A subject identified as suffering from CaP according to the method of the invention may be treated using chemotherapy. Chemotherapy may be systemic or local.

Biotherapy or immunotherapy may also be appropriate.

Treatment of a subject identified using a method of the invention may be carried out in accordance with any of the therapies described above. Thus, any suitable agent can be used to treat such a subject which is known for the treatment of CaP.

Agents which are used in the treatment of CaP may be used in the manufacture of a medicament for use in a method of treatment of a subject identified according to the method of the invention. Thus, the condition of a subject identified as having CaP can be improved by administration of an agent which is used in the treatment of CaP. A therapeutically effective amount of an agent which is used in the treatment of CaP may be given to a patient identified according to a method of the invention.

An agent which is used in the treatment of CaP may be administered in a variety of dosage forms. Thus, they can be administered orally, for example as tablets, troches, lozenges, aqueous or oily suspensions, dispersible powders or granules. The agent which is used to treat CaP may also be administered parenterally, either subcutaneously, intravenously, intramuscularly, intrasternally, transdermally or by infusion techniques. Such an agent may also be administered as a suppository. A physician will be able to determine the required route of administration for each particular patient.

The formulation of an agent used in the treatment of CaP will depend upon factors such as the nature of the exact agent, whether a pharmaceutical or veterinary use is intended, etc. An agent which is to be used to treat CaP may be formulated for simultaneous, separate or sequential use.

Products containing means for determining the absence or presence, or level or expression, of one or more marker gene in a body fluid of a subject and an agent which used in the treatment of CaP as a combined preparation for simultaneous, separate or sequential use in a method of treatment of the human or animal body by therapy are also provided by the invention. Such a product may comprise both means for diagnosis and means for therapy.

An agent used in the treatment of CaP is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of an agent which used in the treatment of CaP is administered to a patient. The dose of an agent which used in the treatment of CaP may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated, the type and severity of the degeneration and the frequency and route of administration. Preferably, daily dosage levels are from 5 mg to 2 g.

The following Examples illustrate the invention:

### EXAMPLES

### Introduction

Blood samples from patients attending the Uro-oncology out patients clinic at St. George's Hospital have been collected. They have been grouped according to diagnosis based on clinical details and results of histopathological analysis as follows:

The NEOM group (no evidence of malignancy) consists of those patients whose biopsy results showed no evidence of malignancy. This group includes patients diagnosed with BPH (benign prostatic hyperplasia) for which they consequently underwent channel TURP (trans-urethral resection of prostate). The median age for this group is 60. The median serum PSA (prostate specific antigen) value at the time of sampling is 5.35 ng/ml. The median PSA at the time of histology is 6.2 ng/ml. The median interval between histology and sampling is 145 days.

The LocCap group (localised cancer) includes those patients who have biopsy proven prostate adenocarcinoma but no clinical and/or radiological evidence of metastatic disease. The median age of this group is 72. The median serum PSA at time of sampling is 7.35 ng/ml. The median serum PSA at the time of histological diagnosis is 12.85 ng/ml. The median time interval between histological diagnosis and sampling is 212 days. This group contains patients undergoing surveillance and those on treatment. Exact data on type of treatment is not available.

The MetCap (metastatic cancer) group consists of patients who demonstrated evidence of widespread disease. The majority of these patients have had positive bone scans. Two were diagnosed to have metastatic disease on Pelvic CT/MRI (computed tomography/magnetic resonance imaging). The median age of this group is 71. The median serum PSA at the time of sampling is 27.6 ng/ml. The median serum PSA at time of histological diagnosis is 244 ng/ml. Median time interval between histology and sampling is 483 days. Patients in this group are all under some form of active treatment. Data on type of treatment is not available.

The RP group (radical prostatectomy) consists of patients who have had a Radical Prostatectomy. All of these samples were taken after the operation (range from 81 to 1584 days after operation). There are no patients who have had a sample taken both pre- and post-operatively. Median time between operation date and sampling is 449 days. Median pre-operative serum PSA is 7.6 ng/ml. Median serum PSA at time of sampling is 0.1 ng/ml and the mean is 0.4 ng/ml. Histology of operative specimens showed positive margins in 10/18 cases. There are 4 patients who showed biochemical recurrence at the time of sampling. This figure increases to 9 when serum PSA data until December 2004 is taken into account. Of these, 3 had negative margins on the operative specimen.

mRNA has been extracted from patient blood samples and transcribed into cDNA, ready for qualitative and quantitative amplification using polymerase chain reaction. The assays are all carried out in quadruplet.

Our research relies on a database set up in 1995 for prospective pathology based prostatic disease for all cases biopsied at St George's or sent in from outside independent hospitals. The data includes information on the method of diagnosis, grade, and stage of any tumour present together with PSA values where available. The database ensures that the diagnosis and clinical details of each patient whose blood is used in the analyses is accurate, thus enabling correlation of our results with the clinical diagnosis.

Expression of prostate-specific, and prostate cancer-specific markers, has been determined. The markers have been prioritised according to their characteristic and potential for use in a diagnosis test.

We have initially established and optimised RT-PCR followed by the establishment and optimisation of relative quantitative RT-PCR using the Light Cycler™ (Roche). Relative quantitative RT-PCR (qRT-PCR) measures, not just the presence of circulating prostate cells, but the actual levels of prostate (cancer) cells in the blood. This is important as it enables the monitoring of disease development and response to therapy. Results have been correlated with existing patho-histological diagnostic data, enabling the sensitivity of the CaP markers in the RT-RCR test to be determined. We have correlated the results obtained using each marker tested with a known stage of disease development.

### Example 1

### E2F3 as a Cap Marker

### Materials and Methods

### Patient Recruitment

Patients attending the Uro-oncology clinic at St. George's Hospital (London, UK) were recruited on the basis of diagnosis by prostate biopsies and transurethral resection of the prostate (TURP). Blood samples were obtained following fully informed consent. The research was carried out in accordance with declaration of Helsinki (2000) of the World Medical Association. Ethical approval for this study was obtained from the Wandsworth Local Research Ethics Committee.

Patients were classified into distinct groups based on clinical diagnosis and histopathological information as well as radiological information (bone scans and CT/MRI scans). Gleason scores for each patient were available. Gleason score, the most commonly used CaP grading system, involves the assignment of numbers to cancerous prostate tissue, ranging from 1 to 5, based on how much the arrangement of cancer cells mimics the way normal prostate cells form glands. Two numbers are assigned to the most common patterns of cells that appear, which are then combined to determine the Gleason score (ranging from 1 to 10).

RNA Extraction and cDNA Synthesis

Total RNA was extracted in quadruplet from blood (100 patients and 10 normal male control individuals) using RNAzol™ (Biogenesis) according to the manufacturer's protocol. Two micrograms of total RNA were reversed transcribed into first-strand cDNA using SuperScript™ and an oligo (dT)₁₂₋₁₈ primer mixture (Invitrogen) according to the manufacturer's protocol.

### Analysis of E2F3 Expression by RT-PCR

Cancer specificity of E2F3 was verified using RT-PCR and mRNA extracted from LnCaP (androgen-sensitive) and PC3 (androgen-insensitive) cell lines and 10 normal male samples. Gene specific primers for E2F3 were designed [5′-aatatggcgtagtatctccg-3′ (forward) and 5′-cttcccaaacatacacccac-3′ (reverse)] based on the published mRNA sequence (accession number: NM_{—}001949). Following first-strand synthesis, E2F3

cDNA was denatured at 95° C. for 15 min, then amplified using 40 cycles of 95° C./1 min, 55° C./1 min and 72° C./1 min. This was followed by a final elongation step of 72° C. for 6 min. PC3 and LnCaP RT-PCR products were electrophoresed on 1% agarose gels and were sequenced to confirm the correct identity of the amplified product. RT-PCR was also carried out using primers for the housekeeping gene, GAPDH (forward: 5′-tgcaccaccaactgctta-3′ and reverse: 5′-ggatgcagggatgatgttc-3′), to determine RNA quality.

### Quantitative RT-PCR

Relative quantitative RT-PCR was carried out for E2F3 and GAPDH genes (primers as before) using a LightCycler™ (Roche) and SYBR Green I according to manufacturer's protocol. Levels of E2F3 mRNA and GAPDH mRNA were calculated by the construction of calibration curves using purified E2F3 PCR product and GAPDH plasmid, respectively. Relative quantification was calculated as a ratio of the amount of target molecule divided by the amount of GAPDH (E2F3/GAPDH). Points from the E2F3 and GAPDH standard curves were included in each patient sample run, to enable accurate calculation of relative quantification.

Melting curve analysis was carried out following quantification to confirm the specificity of the qRT-PCR reaction and to distinguish between specific and non-specific E2F3 products and primer dimers. E2F3 qRT-PCR products were electrophoresed on 1% agarose gels to confirm melting curve analysis results.

### Statistical Analysis

Statistical analyses were carried out using S-plus (Insightful Corp, Seattle, 2003) and SPSS 11.5 (SPSS, Chicago, 2002). Results from the different patient groups were analysed using ANOVA and multiple comparisons for all pair-wise contrasts [S-plus 6.0 for Windows, Guide to Statistics, Vols. I, II (2001) Insightful Corporation, Seattle] of relative E2F3 expression between patient groups. An alternative non-parametric method (Kruskal-Wallis rank sum test) was also used to determine differences in relative E2F3 expression between patient groups. In addition, data were further analysed using a multinomial model. In order to achieve optimal results concerning the inclusion of other covariates, the AIC criterion was taken into account. Gleason score, age and serum PSA were included as covariates to determine their effect on AIC score and hence effect on model suitability.

### Results

### Evaluation of E2F3 Expression in CaP Cell Lines and Normal Male Individuals

E2F3 gene expression was detectable in CaP cell lines, LnCaP and PC3 using RT-PCR. No E2F3 product was found in normal male individuals, thus confirming its cancer specificity. GAPDH expression was positive in both cell lines and normal male individuals, thus verifying mRNA integrity.

### Quantitative E2F3 Expression Profiling in Benign and Malignant Prostate Specimens

Relative quantitative E2F3 gene expression levels were calculated in blood RNA samples taken from patients with benign prostatic hyperplasia (BPH, n=8), localised CaP (LocCaP, n=51), metastatic CaP (MetCaP, n=23) and radical prostatectomy (RP, n=18). Samples were analysed in quadruplet.

Melting curve analysis of all four cancer patient groups showed an 84° C. melting temperature for the E2F3 qRT-PCR product confirming correct identity. qRT-PCR was also carried out on male control samples, despite negative RT-PCR results, to determine whether E2F3 was not expressed or whether expression was below the levels of detection using RT-PCR. However, melting curve analysis did not show any PCR product, confirming that there is no E2F3 expression in normal male control samples.

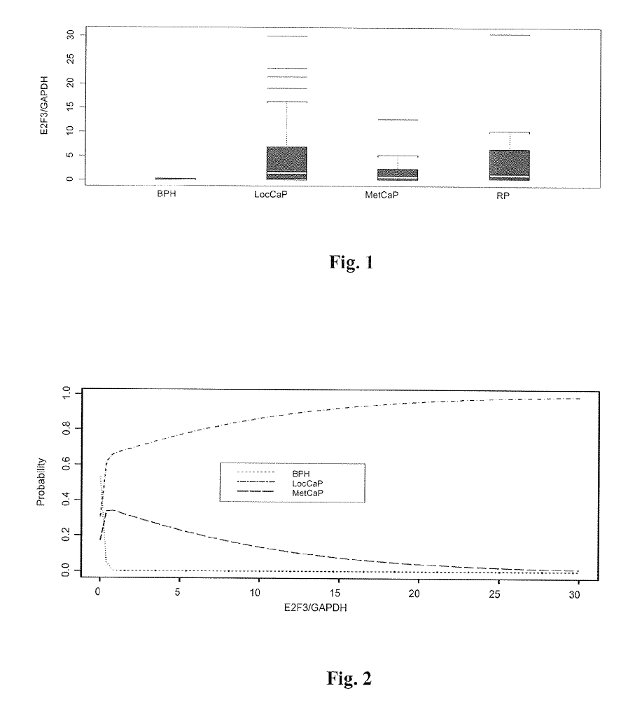

The E2F3 qRT-PCR assay was highly sensitive since levels of E2F3 expression were extremely low in the BPH patient group (mean 0.12, median 0.055). E2F3 expression was found to be massively up-regulated in the LocCaP patient group (mean 4.67, median 1.54). E2F3 levels in the MetCaP patient group (mean 1.89, median 0.68) were lower than that of the LocCaP group but significantly higher to those of the BPH group (FIG. 1). These results indicate that higher levels of E2F3 expression are associated with more aggressive disease stages at least in the case of transition from benign disease to locally invasive CaP. Patients who had undergone radical prostatectomy showed high levels of mean E2F3 expression, similar to those obtained for the LocCaP group. Further analysis of our postoperative clinicopathological data for each individual RP patient was carried out. Of the 14 RP patients, where accurate histopathological information was available, 9 patients presented with positive margins, which would explain the high levels of E2F3 expression. Of the 5 remaining RP patients, who presented with negative margins, 2 demonstrated very low E2F3 expression levels, similar to those obtained for the BPH group. Of the remaining 3 RP patients, one demonstrated high E2F3 expression but post-operative serum PSA levels were extremely high, indicating disease recurrence. The two cases with negative surgical margins but high E2F3 levels may eventually prove to suffer metastatic disease during follow-up as some patients do demonstrate relapse in spite of apparently having had all cancer excised at the time of surgery. Their progress is being monitored.

For the purpose of the multi-comparison method a log transformation was necessary to satisfy the test's assumption for normality. Statistical analysis using the multi-comparison test [S-plus 6.0 for Windows, Guide to Statistics, Vols. I, II (2001) Insightful Corporation, Seattle] showed that there are highly significant differences in E2F3 expression between the different patients groups (p-value<0.001), with the exception of that between the LocCaP and RP patient groups. Similar results were obtained using the Kruskal-Wallis rank sum test (p-value<0.001).

The predicted values derived from a multinomial analysis in Splus were used in the construction of a plot showing the probability that a patient is predicted to belong to each of the three patient groups according to their E2F3 values (FIG. 2). Above E2F3 levels of 2, the distribution of probabilities between diagnosis of LocCaP and the other two patient groups (BPH and MetCaP) becomes more pronounced with the LocCaP group becoming dominant. Therefore, for values of E2F3 above 2, the probability of a diagnosis being on of the LocCaP is consistently above 0.5 Sand in some cases reaching levels close to 1). On the other hand, the probability of the diagnosis of BPH or MetCaP is reduced significantly after the E2F3 threshold level of 2.

At very low E2F3 expression levels (0 to 1), the probability of diagnosis in the three groups cannot be separated. It is worth noticing that high probability levels for LocCaP dominate the upper spectrum of the plot, which inevitably can lead to some misclassification in the case of MetCaP patients (see also diagnostic accuracy section).

The inclusion of Gleason scores substantially decreased the AIC score for the multinomial model (an indicator that balances goodness of fit and over-parameterisation, with small values of AIC pointing towards a more parsimonious model [Burnham and Anderson (2002) Modal Selection and Multimodal Inference. Springer, New York], indicating their contribution to accurate diagnosis (FIG. 3). For the purpose of the multinomial model, patients from the BPH group were considered as having a Gleason score equal to zero. In the case of less aggressive forms of cancer (Gleason=2) the LocCaP patient group can be clearly separated from the MetCaP patient group, in terms of their relative E2F3 values. However, in more aggressive forms of cancer (Gleason=7 and 10), MetCaP patient group increases its likelihood share for a larger interval of E2F3/GAPDH values. In the most aggressive cancers (Gleason=10), diagnosis of MetCaP has a higher probability for relative E2F3 values between 0 and 8.

On inclusion of serum PSA levels and age of diagnosis as covariates, the AIC was increased indicating that they had no effect on delineating probabilities of diagnosis (results not shown).

### Prognostic Accuracy

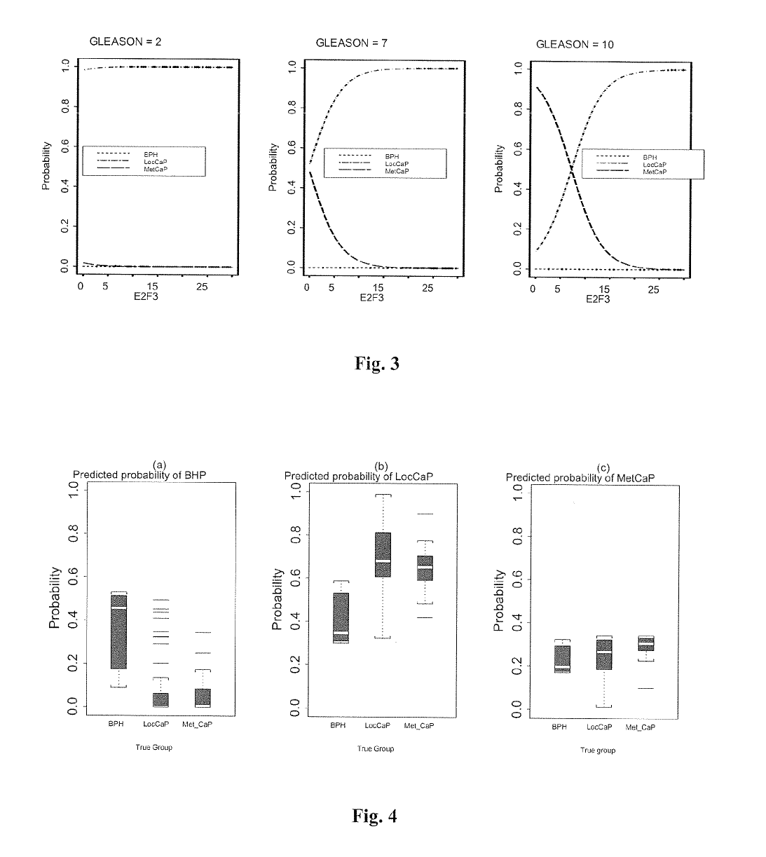

Based on the same statistical model as above it is clear that the prognostic accuracy based on the values of the E2F3 gene depends on the type of patient group it is employed to predict. FIG. 4 shows that accurate results can be achieved when the target is to discriminate between the BPH group and the other two cancer groups (left hand side plot). However, it becomes progressively more difficult to discriminate the various cancer groups from the locCaP to MetCaP. We reiterate our views that this can be due to other mechanisms associated with disease progression from clinically invasive CaP to MetCaP.

As mentioned earlier, high probability levels for LocCaP dominate the upper spectrum of the plot, which inevitably can lead to some misclassification in the case of MetCaP patients. This can be seen in FIG. 4. FIG. 4 shows the distribution of predicted probabilities of being classified in each of the three patient groups according to relative E2F3 expression levels, split according to true diagnosis. E2F3 expression levels clearly discriminate between BPH and malignant disease (FIG. 4a), but are less well able to accurately discriminate between LocCaP and MetCaP (FIGS. 4b and 4c).

### Discussion

Our findings demonstrate that E2F3 can be used as a highly specific marker for the early diagnosis and accurate staging of CaP using the sensitive, non-invasive technique of qRT-PCR.

E2F3 was highly expressed in both LnCaP and PC3 cell lines indicating that E2F3 is one of the several mechanisms involved in prostate carcinogenesis and progression rather than being the end product of CaP. E2F3 expression levels in patients with metastatic CaP were lower than those of the localised CaP patient group but significantly higher than levels in the BPH patient group, indicating that as the disease progresses from clinically localised CaP to androgen-dependent metastatic CaP, there may be a synergistic action between E2F3 and the effect of the androgen receptor (AR). This hypothesis, however, needs to be further investigated (e.g. E2F3 expression studies in CaP cell lines expressing different levels of AR). In addition, further subdivision of the metastatic patient group on the basis of sensitivity/resistance to AR, may be useful in further evaluating the above hypothesis.

The high levels of mean E2F3 expression in the RP group similar to those obtained for the LocCaP group may be indicative of the presence of previously undetected micrometastases. It is well documented that even after surgery, tumour cells are still present in blood circulation, often being a possible reason of disease recurrence and/or metastasis (e.g. presence of minimal amounts of circulating tumour cells is correlated with poor prognosis in colorectal cancer patients) [Schott et al. (1998) Ann. Surg. 227, 372-379].

The use of quantitative RT-PCR for E2F3 together with the multinomial regression model demonstrated that above levels of relative E2F3 expression of 2, there is a higher probability of a patient being diagnosed with clinically invasive CaP. Our model demonstrates the ability to diagnose CaP at the early stages of disease development when treatment is more effective. However, using the probability plot, there are still levels of E2F3 expression for which accurate diagnosis is not possible. This is not surprising since it is well documented that several molecular mechanism and distinct sets of genes, representing distinct biochemical pathways, are involved in disease development and progression. These results further highlight the importance of developing a set of diagnostic gene markers for the early diagnosis and accurate staging of CaP. In addition, E2F3 is part of a control axis (pRB-E2F3-EZH2) that may represent an underlying mechanism of prostate carcinogenesis. E2F3 has been shown to be overexpressed in locally invasive CaP with a decrease in expression levels in MetCaP patients; whereas EZH2 similarly overexpressed in LocCaP is up-regulated further in MetCaP patients. Therefore, evaluation of EZH2 gene expression levels by qRT-PCR and inclusion of the results in our E2F3 probability model will further help in accurately distinguishing not only between benign disease and locally invasive cancer but also between clinically localised and metastatic CaP.

The use of qRT-PCR in the analysis of E2F3 expression in blood of CaP patients could prove to be an accurate and sensitive, non-invasive technique to diagnose and monitor disease development and progression, allowing for more timely and effective therapy on the basis of individual gene expression profiles.

### Example 2

### HIF-1α as a Marker for CAP

### Methods

Patients attending the Uro-oncology clinic at St. George's Hospital (London, UK) were recruited on the basis of diagnosis by prostate biopsy.

Total RNA was extracted in quadruplet from blood taken from 164 patients and 10 normal male control individuals (RNAzol method—Biogenesis) and was reversed transcribed into first-strand cDNA using SuperScript™ II preamplification system with an oligo(dT)12-18 primer mixture (Invitrogen).

Patients were classified into four distinct groups based on clinical diagnosis and histopathological information as well as radiological information (bone scans and CT/MRI scans): No Evidence of Malignancy (NEOM, N=36) also including patients diagnosed with BPH; localised CaP (LocCaP, N=67); metastatic CaP (MetCaP; N=27) and post-operatively obtained blood samples from patients who had undergone radical prostatectomy (RP, N=34).

HIF-1α gene expression was analysed in blood samples of CaP patients by qRT-PCR using a LightCycler™ (Roche) and SYBR Green I.

Data was normalised using a housekeeping gene (GAPDH2) to maintain constant levels between the four groups. Results were expressed as HIF-1α/GAPDH2.

### Results

### Validation Experiments of HIF-1α Expression

### 1. Amplification Efficiencies and Standard Curve

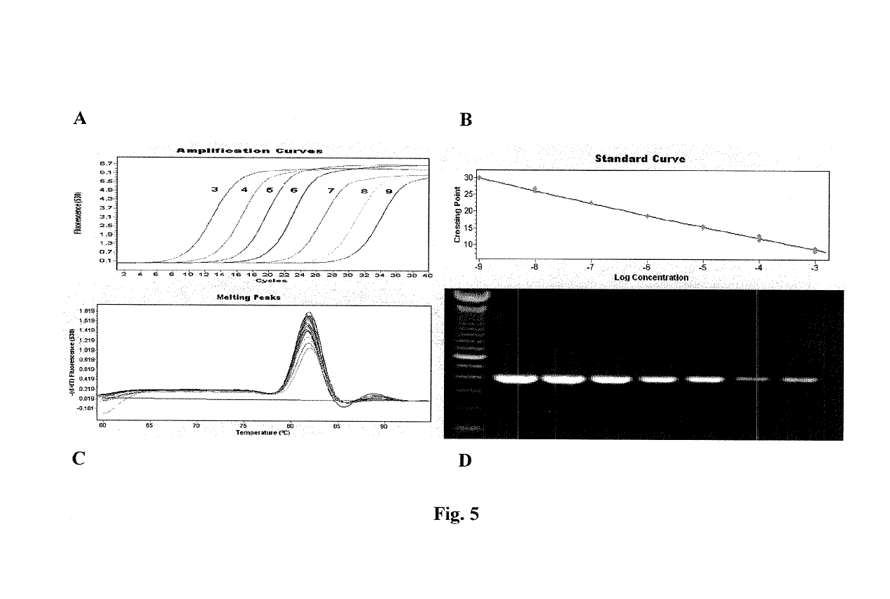

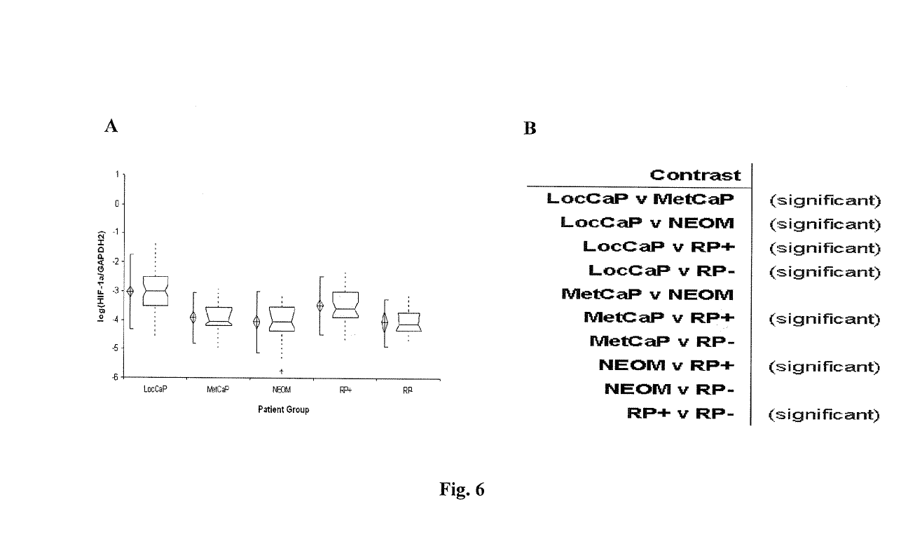

The reaction for HIF-1α was both highly reproducible (FIG. 5A) and sensitive (9 logs of magnitude FIG. 5B). The standard curve showed a linear response with a correlation coefficient (R²) of 0.9985 and slope of −3.53 (FIG. 5B), thus demonstrating an exponential amplification efficiency of 1.995 and reaction efficiency of 92% (Efficiency=10^{[-1/slope]}−1)

### 2. Melting Curve Analysis

Melting curve analysis demonstrated the amplification of a single product with a distinct narrow peak (FIG. 5C) indicating a highly specific reaction. Further reaction specificity was confirmed by agarose gel electrophoresis (FIG. 5D) and sequencing of amplicons.

### 3. Statistical Analysis

The RP group was further divided into patients with post-operatively positive surgical margins (RP⁺) indicative of residual disease and those with negative margins (RP-) to determine any prognostic implication of HIF-1α in monitoring disease relapse. The results are shown in FIG. 3.

Significant differences were found in relative HIF-1α expression levels (HIF-1α/GAPDH2) between the different patients groups (p<0.0001) with the exception of that between MetCaP and NEOM, NEOM and RP-(negative margins) and MetCaP and RP-.

TABLE 1
Comparable statistics (linear scale) of quantitative
HIF-1α expression in the four patient groups

| Patient Group | NEOM (n = 28) | LCaP (n = 63) | MCaP (n = 25) | RP (n = 30) |
|---|---|---|---|---|
| Mean (×10⁻⁴) | 1.877 | 37.20 | 2.342 | 8.426 |
| 95% of Mean (×10⁻⁴) | 1.029 to 2.725 | 16.17 to 58.22 | 1.091 to 3.593 | 3.935 to 12.92 |
| Median (×10⁻⁵) | 8.75 | 100.6 | 8.545 | 26.18 |

### Discussion

HIF-1α was found to be over-expressed in 59/63 of LocCaP patients (20-fold mean increase in gene expression levels compared to the baseline population of NEOM; p<0.0001) suggesting that hypoxia driven by HIF-1α up-regulation is an early event in CaP formation.

HIF-1α down-regulation in the MetCaP patient group (p<0.0001 between LocCaP and MetCaP) indicates that after the formation of neovasculature and the establishment of new blood vessels, tumour tissue is not a hypoxic environment and that an alternative mechanism is necessary for the invasion of tumour cells into the bloodstream and the formation of secondary metastatic sites.

High levels of HIF-1α expression were found in the RP patient group. Of these, a high proportion was shown to have positive margins suggesting the presence of residual disease and highlighting the need for continued monitoring and possible additional therapy.

qRT-PCR analysis of HIF-1α expression in blood of CaP patients is a sensitive, non-invasive technique to diagnose and monitor early stages of disease development. These results further suggest that HIF-1α may become a potential target for CaP therapy through the development of new agents that inhibit angiogenesis and tumour growth via inhibition of its expression.

### Example 3

### CAXII as a Marker for CAP

qRT-PCR Results

All patient samples were positive for CAXII expression. Variations in signal intensity indicated differences in the actual levels of the enzyme among patients. Quantitative RT-PCR showed that:
CAXII expression is up-regulated in the localised cancer group (LCaP) (6-fold) compared to the benign prostatic hyperplasia (BPH) group; and
down-regulated in the MCaP (metastatic cancer) group (the majority being hormone-refractory) (18-fold), compared to the LCaP group.

TABLE 2
Summary of CAXII qRT-PCR Results
RT-PCR: positive expression

| BPH | LCaP | MCaP | Male controls |
|---|---|---|---|
| 5/7 | 66/70 | 31/35 | 0 |

qRT-PCR: mean expression levels

| n = 10 | n = 8 | n = 18 |
|---|---|---|
| 118.5 | 703.9 | 40.1 |

### CONCLUSION

CAXII is hypoxia-induced (via HIF-1) and so the results indicate that hypoxia is a mechanism involved in CaP development. However, down-regulation of CAXII in the MCaP patients indicates a possible alternative pathway that overrides the hypoxia-induced mechanism in hormone-refractory end-stage CaP. These characteristics of CAXII indicate that it may actually be involved in the early development of prostate cancer by changing the environment of the tumour.

CAXII is a potentially excellent molecular marker in routine clinical diagnosis and prognosis of CaP.

### Example 4

### Analysis of Multiple Markers

Expression of the following markers in RNA extracted from patient blood samples was determined by qRT-PCR as described above using the primers shown in Table 3: e-cad, RECK, caveolin, MTSP-1, HIF-1α, E2F3, Clusterin, MMP9, MMP15, MMP24, PIM-1, IGFBP-2, IGFBP-3 and E2F4. The expression data was analysed by various statistical techniques and the results are summarised in Tables 4 to 8.

Table 4 shows the results of a descriptive analysis of the levels of marker expression in patients with no evidence of malignancy (NM), localised CaP (LC) and metastatic cancer (MC). The mean and median expression levels of each marker in each patient group is shown together with the standard deviation/interquartile range (SD/IQR) and the confidence limits (95% Cl). The results of the 1-way ANOVA test are also shown together with an indication of whether the markers are up- or down-regulated. A cross indicates that no significant differences in the levels of marker expression between the indicated groups were observed.

Table 5 shows the results of ROC/AUC analysis to determine the ability of each marker to discriminate between prostate cancer (cancer) and non-cancer (benign) patients. The analysis was carried out as previously described (Metz et al. 1978). The Table shows the number of patients in each group in which each of the markers were analysed, the area under the curve (AUC), the confidence limits (95% Cl), the ability of each marker to distinguish cancer and benign patients (Classification) and p-value. For the markers with discriminatory power, the Table also shows the positive predictive value (PPV), negative predictive value (NPV), efficiency, sensitivity and specificity of each marker.

Tables 6 and 7 show the results of ROC/AUC analysis to determine the ability of each marker to discriminate between patients with no evidence of malignancy (NM) and localised CaP (LC), NM and metastatic cancer (MC) and LC and MC. The analysis was carried out as previously described (Metz et al. 1978). Table 6 shows the area under the curve (AUC), the confidence limits (95% Cl) and p-value. Table 7 further indicates the number of patients (n) in each group in which each of the markers was analysed and the positive predictive value (PPV), negative predictive value (NPV), efficiency, sensitivity and specificity of each marker.

Table 8 shows the results of a stepwise discriminant analysis used to investigate the diagnostic strength of selected markers when used in combination to distinguish cancer patients from non-cancer patients. The analysis was carried out as previously described (Abramowitz and Stegun, (Eds.), Handbook of Mathematical Functions with Formulas, Graphs and Mathematical Tables, 9^{th} printing, New York: Dover, 927-928, 1972; Feinstein, Multivariable Analysis, New Haven, Conn.: Yale University Press, 1996; Gould, The Mismeasure of Man, rev. exp. ed. New York: W. W. Norton, 1996; Hair, Multivariate Data Analysis with Readings, 4^{th} ed. Englewood Cliffs: Prentice-Hall, 1995; Schafer, Analysis of Incomplete Multivariate Data. Boca Raton, Fla.: CRC Press, 1997; and Sharma, Applied Multivariate Techniques, New York: Wiley, 1996).

TABLE 3
Primers used for CaP marker PCR

| | | | |
|---|---|---|---|
| | | | Alt splicing? |
| Marker | Primer sequence | Position in exons | (y/n) |

| | | | | |
|---|---|---|---|---|
| E2F3 | F | AATATGGCGTAGTATCTCCG | Exon 7 | — |
| | R | CTTCCCAAACATACACCCAC | (334 bp) | |
| EZH2 | F | AATTTCCGAGGTGGGC | 14 and 16 | No |
| | R | GAAAGTACACGGGGATAG | | |
| c-met | F | GCAGTGCAGCATGTAGTGAT | 7 and 9 | No |
| (MET) | R | CAGGAGCGAGAGGACATTGG | (238 bp) | |
| FAS | F | TTTCTGCTCCTGCACACACT | 23 and 26 | Possible |
| | R | AGGTGCTGCTGAGGTTGGAGA | (420 bp) | |
| AMACR | F | CGTATGCCCCGCTGAATCTCGT | 3 and 5 | No |
| | R | TGGCCAATCATCCGTGCTCATC | | |
| HIF-1α | F | CGCATCTTGATAAGGCCTCT | F @ exon 2; | No |
| | R | TACCTTCCATGTTGCAGACT | Reverse @ exons | |
| | | | 5 & 6 | |
| | | | (418 bp) | |
| CAIX | F | CCGAGCGACGCAGCCTTTGA | 8 and 11 | Possible |
| | R | AGGTAGCCGAGACTGGAGCCTAG | (256 bp) | |
| CAXII | F | GGACAAATGGGGACAGGAAGGATCAAG | Exon 11 | — |
| | R | GAGGACATTTCATGCTGTCAAAATGAG | (893 bp) | |
| Hepsin | F | TGTCCCGATGGCGAGTGTT | F @ exon 10; | No |
| | R | CCTGTTGGCCATAGTACTGC | Reverse @ exons | |
| | | | 11 & 12 | |
| | | | (282 bp) | |
| PIM-1 | F | ATCAGGGGCCAGGTTTTCT | 5 and 6 | No |
| | R | AAAGGCTGCTATTTGCTGGG | (206 bp) | |
| PSGR | F | GCCACCTGTGTGCTTATTGGTATCC | Both @ exon 2 | — |
| | R | GACACAATAGGAGTGCGAGAGGACATTG | (518 bp) | |
| JAGGED-1 | F | CCTATACGTTGCTTGTGGAGG | 3 and 6 | No |
| | R | TGCCAGGGCTCATTACAGAT | 450 bp | |
| Caveolin | F | TCGCCATTCTCTCTTTCCTGCACA | 3 and 3 | No |
| | R | TGGAATAGACACGGCTGATGCACT | | |
| e-cad | F | AAGAAGCTGGCTGACATGTACGGA | 16 and 16 | No |
| | R | AACCACCAGCAACGTGATTTCTGC | | |
| hk2 | F | TACCACCCTGGGGTTATGAA | 5 and 5 | |
| | R | TAGTAACAAGACGGTGGGGC | | |
| hk3 | F | CCAGACACTCACAGCAAGGA | 5 and 5 | |
| | R | GTCCTCCAGACAACCCTCAG | | |
| EF-1A | F | CATGCTGGAGCCAAGTGCTA | 4 and 6 | No |
| | R | GCCAACAGGAACAGTACCAATA | (177 bp) | |
| MMP2 | F | ACTGCTGGCTGCCTTAGAAC | 13 and 13 | No |
| | R | TGAACAGGGGAACCATCACT | | |
| MMP24 | F | AGTCCAGGAATGGGTGTGAG | 9 and 9 | No |
| | R | CCACCACTTCAGCTGTACGA | | |
| MMP9 | F | AGTTCCCGGAGTGAGTTGA | 13 and 13 | No |
| | R | CACCTCCACTCCTCCCTTTC | (200 bp) | |
| MMP15 | F | AACTGGCTGCGGCTTTATGG | 2 and 3 | No |
| | R | AGGTCAGATGGTGGTTGTTCC | (274 bp) | |
| E2F4 | F | CACCAAGTTCGTGTCCCTTC | 1 and 2 | No |
| | R | GCCCGATACCTTCCAAAACA | (130 bp) | |
| MTSP-1 | F | CTACCACAAGGAGTCGGCT | 4 and 6 | No |
| | R | TGTCCTGGGTCCTCTGTACT | (227 bp) | |
| RECK | F | GGATAACCAAATGTGCCGTG | 2 and 5 | No |
| | R | CAATAGCCAGTTCACAGCAG | (203 bp) | |
| IGFBP-2 | F | TATGAAGGAGCTGGCCGTGTT | 2 and 3 | No |
| | R | CAGGCCATGCTTGTCACAGT | (248 bp) | |
| IGFBP-3 | F | TGCCGTAGAGAAATGGAAGAC | 3 and 4 | No |
| | R | TAGCAGTGCACGTCCTCCTT | (221 bp) | |
| Clusterin | F | TCCAGGACAGGTTCTTCACC | 5 and 5 | |
| | R | TGCTGAGCCTCGTGTATCAT | | |

### Table 4: Descriptive Analysis of all Markers

TABLE 4
Descriptive Analysis of all Markers Continued

| | Descriptive Statistics | | | | | | | 1-way ANOVA | | |
|---|---|---|---|---|---|---|---|---|---|---|
| | Mean | | | Median | | | SD/IQR | NM vs. | LC vs. | NM vs. |
| Marker | NM | LC | MC | NM | LC | MC | 95% Cl | LC | MC | MC |
| MMP15 | 4.91E−07 (7) | 2.48E−07 (11) | 9.41E−08 (12) | 2.30E−07 | 2.10E−07 | 9.50E−08 | SD/IQR | x | x | x |
| n = 30 | 6.80E−07 | 2.00E−07 | 5.72E−08 | 5.15E−07 | 2.95E−07 | 6.50E−08 | 95% Cl | | | |
| | 1.37E−07 to | 1.14E−07 to | 5.78E−08 to | 1.00E−08 to | 1.00E−08 to | 4.00E−08 to | | | | |
| | 1.12E−06 | 3.83E−07 | 1.30E−07 | 1.93E−06 | 4.90E−07 | 1.30E−07 | | | | |
| MMP24 | 1.09E−01 (8) | 9.33E−02 (20) | 5.76E−02 (16) | 9.83E−02 | 6.61E−02 | 3.68E−02 | SD/IQR | x | x | x |
| n = 44 | 6.06E−02 | 7.89E−02 | 4.41E−02 | 7.59E−02 | 9.38E−02 | 5.67E−02 | 95% Cl | | | |
| | 5.79E−02 to | 5.63E−02 to | 3.40E−02 to | 3.06E−02 to | 4.02E−02 to | 2.06E−02 to | | | | |
| | 1.59E−01 | 1.30E−01 | 8.11E−02 | 1.59E−01 | 1.01E−01 | 8.93E−02 | | | | |
| PIM-1 | 8.69E−02 (26) | 1.61E−01 (57) | 5.38E−02 (22) | 9.25E−02 | 9.97E−02 | 5.18E−02 | SD/IQR | significant | significant | x |
| n = 105 | 3.87E−02 | 1.40E−01 | 2.29E−02 | 4.71E−02 | 1.99E−01 | 2.50E−02 | 95% Cl | [symbol] | [symbol] | [symbol] |
| | 7.12E−02 to | 1.23E−01 to | 4.37E−02 to | 6.19E−02 to | 5.94E−02 to | 3.83E−02 to | | | | |
| | 1.02E−01 | 1.98E−01 | 6.40E−02 | 1.07E−01 | 1.77E−01 | 6.52E−02 | | | | |
| IGFBP-2 | 1.65E−04 (6) | 6.51E−05 (10) | 1.04E−04 (11) | 1.60E−04 | 5.75E−05 | 8.52E−05 | SD/IQR | x | x | x |
| n = 27 | 1.41E−04 | 3.16E−05 | 6.32E−05 | 1.95E−04 | 4.48E−05 | 8.47E−05 | 95% Cl | | | |
| | 1.68E−05 to | 4.25E−05 to | 6.17E−05 to | 1.67E−05 to | 3.18E−05 to | 3.18E−05 to | | | | |
| | 3.14E−04 | 8.77E−05 | 1.47E−04 | 3.14E−04 | 1.05E−04 | 1.47E−04 | | | | |
| IGFBP-3 | 3.42E−05 (8) | 4.14E−05 (10) | 1.81E−05 (11) | 2.82E−05 | 3.37E−05 | 1.60E−05 | SD/IQR | x | significant | x |
| n = 29 | 2.63E−05 | 2.16E−05 | 1.37E−05 | 2.99E−05 | 2.00E−05 | 1.25E−05 | 95% Cl | [symbol] | [symbol] | [symbol] |
| | 1.23E−05 to | 2.59E−05 to | 8.87E−06 to | 1.05E−05 to | 1.95E−05 to | 6.68E−06 to | | | | |
| | 5.62E−05 | 5.68E−05 | 2.72E−05 | 8.98E−05 | 6.80E−05 | 2.50E−05 | | | | |
| E2F4 | 1.75E−05 (29) | 9.05E−05 (53) | 9.47E−06 (23) | 1.31E−05 | 2.72E−05 | 7.30E−06 | SD/IQR | significant | significant | x |
| n = 105 | 1.61E−05 | 1.91E−04 | 7.55E−06 | 1.94E−05 | 5.46E−05 | 1.06E−05 | 95% Cl | | | |
| | 1.13E−05 to | 3.79E−05 to | 6.21E−06 to | 5.93E−06 to | 1.55E−05 to | 3.87E−06 to | | | | |
| | 2.36E−05 | 1.43E−04 | 1.27E−05 | 2.34E−05 | 5.09E−05 | 1.21E−05 | | | | |

TABLE 5
ROC/AUC Analysis and Diagnostic Screening: Benign vs. Cancer

| | Be- | Can- | | | Classifi- | | | | | | | | | |
|---|---|---|---|---|---|---|---|---|---|---|---|---|---|---|
| Marker | nign | cer | AUC | 95% Cl | cation | p-value | PPV | | NPV | | Efficiency | | Sensitivity | Specificity |
| RECK | 11 | 27 | 0.923 | .830 to 1.0 | Excellent | <0.0001 | 90% | (26/29) | 89% | (8/9) | 89.5% | (34/38) | 96.30% | 72.70% |
| Clusterin | 7 | 41 | 0.927 | .809 to 1.0 | Excellent | <0.0001 | 95.3 | (41/43) | 100% | (5/5) | 96% | (46/48) | 100% | 71.40% |
| MMP9 | 9 | 21 | 0.862 | .730 to .995 | Good | <0.0001 | 85.7% | (18/21) | 66.7% | (6/9) | 80% | (24/30) | 85.70% | 66.70% |
| E2F3 | 16 | 64 | 0.856 | .764 to .949 | Good | <0.0001 | 89.2% | (58/65) | 60% | (9/15) | 83.75% | (67/80) | 90.60% | 56.30% |
| MTSP-1 | 11 | 24 | 0.799 | .642 to .956 | Fair | <0.0001 | 86.9% | (20/23) | 66.6% | (8/12) | 80% | (28/35) | 83.30% | 72.70% |
| HIF-1α | 25 | 74 | 0.778 | .681 to .875 | Fair | <0.0001 | 82.9% | (68/82) | 64.7% | (11/17) | 80% | (79/99) | 91.00% | 44% |
| e-cad | 13 | 35 | 0.763 | .599 to .927 | Fair | 0.0009 | 82.5% | (33/40) | 75% | (6/8) | 81.25% | (39/48) | 94.30% | 46.20% |

| | | | | | | | |
|---|---|---|---|---|---|---|---|
| MMP24 | 8 | 36 | 0.688 | .491 to .884 | Poor | 0.0309 | No significant discriminatory power |
| MMP15 | 7 | 23 | 0.624 | .341 to .908 | Poor | 0.1953 | (p-value and AUC) to distinguish |
| IGFBP-2 | 6 | 21 | 0.611 | .264 to .958 | Poor | 0.265 | between benign and malignant disease |
| E2F4 | 29 | 76 | 0.59 | .477 to .702 | Fail | 0.0594 | (Localised and Metastatic CaP) |
| IGFBP-3 | 8 | 21 | 0.554 | .310 to .797 | Fail | 0.3332 | |
| Caveolin | 14 | 41 | 0.509 | .323 to .694 | Fail | 0.4633 | |
| PIM | 26 | 79 | 0.505 | .386 o .623 | Fail | 0.4679 | |

TABLE 6
Receiver Operator Characteristic/Area Under Curve (ROC/AUC) Analysis

| | NM vs. LC | | | NM vs. MC | | | LC vs. MC | | |
|---|---|---|---|---|---|---|---|---|---|
| Marker | AUC | p-value | 95% Cl | AUC | p-value | 95% Cl | AUC | p-value | 95% Cl |
| e-cad | 0.78 | 0.0005 | .614 to .945 | 0.734 | 0.0106 | .535 to .932 | 0.587 | 0.1981 | .385 to .789 |
| RECK | 0.86 | <0.0001 | .706 to 1.0 | 0.981 | <0.0001 | .935 to 1.0 | 0.857 | <0.0001 | 0.719 to .995 |
| Caveolin | 0.593 | 0.1854 | .390 to .795 | 0.704 | 0.0206 | .508 to .900 | 0.796 | <0.0001 | .652 to .940 |
| MTSP-1 | 0.678 | 0.0547 | .460 to .897 | 0.883 | <0.0001 | .75 to 1.0 | 0.729 | 0.0141 | .524 to .933 |
| HIF-1α | 0.883 | <0.0001 | .808 to .958 | 0.546 | 0.2927 | .380 to .712 | 0.87 | <0.0001 | .789 to .950 |
| E2F3 | 0.879 | <0.0001 | .792 to .967 | 0.803 | <0.0001 | .657 to .948 | 0.691 | 0.0019 | .562 to .821 |
| Clusterin | 0.897 | <0.0001 | .736 to 1.0 | 0.973 | <0.0001 | .912 to 1.0 | 0.823 | <0.0001 | .691 to .954 |
| MMP9 | 0.812 | 0.0003 | .634 to .990 | 0.944 | <0.0001 | .841 to 1.0 | 0.788 | 0.0035 | .579 to .998 |
| MMP15 | 0.571 | 0.32 | .272 to .871 | 0.673 | 0.1315 | .370 to .975 | 0.723 | 0.0324 | .486 to .961 |
| MMP24 | 0.631 | 0.1299 | .403 to .860 | 0.758 | 0.0073 | .551 to .965 | 0.669 | 0.0354 | .486 to .852 |
| PIM-1 | 0.59 | 0.0768 | .466 to .713 | 0.75 | 0.0727 | .608 to .892 | 0.739 | <0.0001 | .630 to .849 |
| IGFBP-2 | 0.633 | 0.2282 | .283 to .984 | 0.591 | 0.308 | .236 to .946 | 0.655 | 0.1061 | .412 to .897 |
| IGFBP-3 | 0.6 | 0.2442 | .317 to .883 | 0.693 | 0.0613 | .448 to .938 | 0.873 | <0.0001 | .717 to 1.0 |
| E2F4 | 0.702 | 0.0002 | .590 to .814 | 0.67 | 0.0114 | .524 to .817 | 0.819 | <0.0001 | .725 to .913 |

TABLE 7
Receiver Operator Characteristic/Area Under Curve (ROC/AUC) Analysis

| | | Diagnostic Screening | | |
|---|---|---|---|---|
| Marker | | NM vs. LC | NM vs. MC | LC vs. MC |
| e-cad | ROC/AUC | 0.78 | 0.734 | 0.587 |
| NM = 13 | Cut-off | | | FAIL |
| LC = 22 | PPV | 76.9% (20/26) | 75% (9/12) | [symbol] |
| MC = 13 | NPV | 77.8% (7/9) | 71.4% (10/14) | |
| | Efficiency | 77.1% (27/35) | 73% (19/26) | |
| RECK | ROC/AUC | 0.86 | 0.981 | 0.857 |
| NM = 11 | Cut-off | | | |
| LC = 13 | PPV | 80% (12/15) | 93.3% (10/15) | 83.3% (10/12) |
| MC = 14 | NPV | 89% (8/9) | 100% (10/10) | 73.3% (11/15) |
| | Efficiency | 83.3% (20/24) | 96% (24/25) | 78% (21/27) |
| Caveolin | ROC/AUC | 0.593 | 0.704 | 0.796 |
| NM = 14 | Cut-off | FAIL | | |
| LC = 27 | PPV | [symbol] | 77.8% (7/9) | 52.4% (11/21) |
| MC = 14 | NPV | | 63.2% (12/19) | 85% (17/20) |
| | Efficiency | | 67.8% (19/28) | 68.3% (28/41) |
| MTSP-1 | ROC/AUC | 0.678 | 0.883 | 0.729 |
| NM = 11 | Cut-off | POOR | | |
| LC = 10 | PPV | | 81.25% (13/16) | 81.8% (9/11) |
| MC = 14 | NPV | | 88.9% (8/9) | 61.5% (8/13) |
| | Efficiency | | 84% (21/25) | 70.8% (17/24) |
| HIF-1α | ROC/AUC | 0.883 | 0.546 | 0.87 |
| NM = 25 | Cut-off | | FAIL | |
| LC = 51 | PPV | 83.60% | | 87.20% |
| MC = 23 | NPV | 76.20% | [symbol] | 63% |
| | Efficiency | 81.60% | | 78.40% |
| E2F3 | ROC/AUC | 0.879 | 0.803 | 0.691 |
| NM = 16 | Cut-off | | | POOR |
| LC = 45 | PPV | 85% | 71.40% | |
| MC = 19 | NPV | 69.20% | 71.40% | [symbol] |
| | Efficiency | 82% | 71.40% | |
| Clusterin | ROC/AUC | 0.897 | 0.973 | 0.823 |
| NM = 7 | Cut-off | | | |
| LC = 25 | PPV | 92.30% | 100% | 68.40% |
| MC = 16 | NPV | 83.30% | 94.10% | 86.40% |
| | Efficiency | 90.60% | 95.70% | 78.00% |
| MMP9 | ROC/AUC | 0.812 | 0.944 | 0.788 |
| NM = 9 | Cut-off | | | |
| LC = 13 | PPV | 75% | 80% | 77% |
| MC = 8 | NPV | 80% | 100% | 67% |
| | Efficiency | 76.20% | 88.20% | 73% |
| MMP15 | ROC/AUC | 0.571 | 0.673 | 0.723 |
| NM = 7 | Cut-off | FAIL | POOR | |
| LC = 11 | PPV | [symbol] | [symbol] | 71.4% (10/14) |
| MC = 12 | NPV | | | 77.8% (7/9) |
| | Efficiency | | | 73.9% (17/23) |
| MMP24 | ROC/AUC | 0.631 | 0.758 | 0.669 |
| NM = 8 | Cut-off | POOR | | POOR |
| LC = 20 | PPV | | 85.7% (12/14) | [symbol] |
| MC = 16 | NPV | | 60% (6/10) | |
| | Efficiency | | 75% (18/24) | |
| PIM-1 | ROC/AUC | 0.59 | 0.75 | 0.739 |
| NM = 26 | Cut-off | FAIL | | |
| LC = 57 | PPV | | 66.70% | 43.8% (21/48) |
| MC = 22 | NPV | [symbol] | 81.00% | 96.8% (30/31) |
| | Efficiency | | 72.90% | 64.6% (51/79) |
| IGFBP-2 | ROC/AUC | 0.633 | 0.591 | 0.655 |
| NM = 6 | Cut-off | POOR | FAIL | POOR |
| LC = 10 | PPV | | | |
| MC = 11 | NPV | [symbol] | [symbol] | [symbol] |
| | Efficiency | | | |
| IGFBP-3 | ROC/AUC | 0.600 | 0.693 | 0.873 |
| NM = 8 | Cut-off | POOR | POOR | |
| LC = 10 | PPV | [symbol] | [symbol] | 83.3% (10/12) |
| MC = 11 | NPV | | | 88.8% (8/9) |
| | Efficiency | | | 85.7% (18/21) |
| E2F4 | ROC/AUC | 0.702 | 0.670 | 0.819 |
| NM = 29 | Cut-off | | POOR | |
| LC = 53 | PPV | POOR | [symbol] | 62.50% |
| MC = 23 | NPV | | | 85% |
| | Efficiency | | | 77.60% |

TABLE 8
Stepwise Discriminant Analysis - Diagnostic
Strength of Markers when used in Combination

| | | Cancer | Non-cancer | Overall |
|---|---|---|---|---|
| Starting Markers | Markers used | % correct | % correct | (no. of patients) |
| RECK, Clusterin | RECK | 100 | 100 | 100 (13) |
| RECK, MTSP1 | RECK | 89 | 73 | 84 (38) |
| Clusterin, MTSP1 | Clusterin, MTSP1 | 100 | 100 | 100 (13) |
| RECK, | RECK | 100 | 100 | 100 (13) |
| Clusterin, MTSP1 | | | | |
| RECK, MMP24 | RECK | 100 | 100 | 100 (14) |
| RECK, MMP15/RECK | RECK | 91 | 67 | 86 (28) |
| Clusterin, MMP24 | Clusterin | 82 | 86 | 83 (46) |
| Clusterin, | Clusterin | 100 | 100 | 100 (9) |
| MMP15/RECK | | | | |
| MTSP1, MMP24 | MTSP1, MMP24 | 90 | 75 | 86 (14) |
| MTSP1, MMP15/RECK | MTSP1, MMP15/RECK | 91 | 67 | 86 (28) |

With reference to the results of ROC/AUC analysis determining diagnostic strength of the markers shown in Table 5, RECK and Clusterin are excellent markers with an extremely high combination of sensitivity and specificity that lead to cancer/non-cancer diagnosis and MMP9, E2F3, MTSP-1, HIF-1α and e-cad are good to fair markers.

With reference to Table 8 which shows the results of forward stepwise discriminant analysis, the strongest marker combinations for differentially diagnosing cancer and non-cancer when used in combination involve RECK, Clusterin and MTSP1. Combinations of these markers give an excellent percentage correct diagnosis of cancer and non-cancer (i.e. 100% correct diagnosis for both cancer and non-cancer).

Table 6 illustrates the strength of diagnostic power of markers at different stages of disease development as determined using Receiver Operator Characteristic/Area Under Curve (ROC/AUC) Analysis. The results show that:
RECK, Clusterin and HIF-1α may be used to distinguish between LocCap and MetCap as well distinguishing between NEOM and LocCap;
in addition to RECK, Clusterin and HIF-1α, E2F3 and MMP9 are good markers for distinguishing between NEOM and localised disease and e-cad and E2F4 may also be used for diagnosis at this stage; and
in addition to RECK, Clusterin and HIF-1α, good markers for distinguishing between localised and mestastatic cancer include IGFBP-3 and E2F4. Caveolin, MMP9, PIM-1 and MTSP1 may also be used for this purpose.

The strength of the markers may be further increased by combining their use. Accurate discrimination between MetCap and BPH/NEOM may facilitate monitoring possible relapse post surgery (radical prostatectomy (RP)) and/or effectiveness of CaP therapy. Patients with localised cancer will undergo hormone treatment, radiotherapy and/or surgery (RP). If expression levels of a marker are shown to increase during hormone treatment and/or radiotherapy, this indicates a continuing risk of the cancer developing to the dangerous metastatic stage, thus dictating alternative or more aggressive therapy. Conversely, if therapy is successful, the levels of marker expression would become similar to that given by BPH/NEOM patient samples. Similarly, following RP, if marker expression levels continue to increase or show no signs of decreasing, this may indicate either residual disease or previously undetected metastases (early stage metastatic disease involves the formation of bone micrometastatses, which may not be detected through bone scan).

With reference to the discriminant analysis between NEOM and MetCap in Table 6, RECK, Clusterin and MMP9 are excellent markers for potential use in monitoring response to therapy and MTSP1 and E2F3 are good markers. All markers showed highly significant differences between NEOM/BPH and MetCap patient groups.

### Example 5

### Alternative Splicing

Alternative splicing is a well documented phenomenon that is frequently associated with the neoplastic transformation (Roy et al., Nucleic Acids Res. 2005 33(16):5026-33, Okumura M et al., Biochem Biophys Res Commun. 2005 334(1):23-9, Schwerk C et al., Mol. Cell. 2005 19(1):1-13). The underlying mechanism involves mutations (substitutions, deletions or insertions) at the intron/exon boundaries which destroy the recognition site, or within exons or introns which creates an additional recognition site for subsequent splicing. In this way, additional material may be spliced into the mRNA, or material may be spliced out of the mRNA, resulting in larger or smaller products than those expected or calculated on RT-PCR (reverse transcriptase polymerase chain reaction).

These alterations in the mRNA and, therefore, the translated protein, may be a precurser to tumour formation. As such, monitoring changes in mRNA splicing has the potential of diagnosing risk of tumour development (Atanelov et al., J. Gastroenterol. 2005 40 Suppl 16:14-20, Kirschbaum-Slager N et al., Physiol Genomics. 2005 21(3):423-32). Several markers have been reported as demonstrating alternatively spliced forms in prostate cancer (Mubiru J N et al., Prostate. 2005 65(2):117-23, Stavropoulou P et al., Clin Chim Acta. 2005 357(2):190-5).

The technique to determine the presence or absence of these tumour precursor mutations and alternative splicing involves RT-PCR using mRNA extracted from blood samples and primers designed specifically to the nucleotide sequences flanking the regions carrying the splice mutations. Reaction products are electrophoresed using agarose gels and product sizes analysed. Additional products are easily demonstrated. Determination of the additional splice product in an individual at an early stage of the disease (BPH) is indicative of subsequent tumour development.

The cancer specific, prostate specific or prostate cancer specific markers, EZH2, e-cad and CAIX showing evidence of alternative splicing were investigated further. The primer sequences used and the results are shown in Table 9.

TABLE 9
Markers that show alternative splicing

| | | | | | Correct | | Additional | Additional products | |
|---|---|---|---|---|---|---|---|---|---|
| | | | | Expected | product in cell | | products in | in cell lines and | |
| | | | Position of | product | lines? | | patient samples? | patient samples? | |
| | Sequences | | primers | size | (y/n) | | If so, give | If so, give | |
| Marker | of Primers | | (exons) | (bp) | LnCap | PC3 | product size(s) | product sizes | Additional notes |
| EZH2 | F | tacctggctg | 14 | 327 | Y | Y | 390 | Normal males not | Additional product |
| | | tccgag | | | | | | done | seen in all patient |
| | R | gatgcaaccc | 17 | | | | | Some patients in | groups |
| | | gcaagg | | | | | | all groups incl | |
| | | | | | | | | BPH/NEOM | |
| e-cad | F | actacttgaa | 16 | 616 | Y | Y | | Yes, both cell lines | Pt groups not done as |
| | | cgaatgg | | | | | | (300 bp) | additional product in |
| | R | ttagtcatg | 16 | | | | | | cell lines |
| | | cgtagtg | | | | | | | |
| CAIX | F | CCGAGCGACGCA | 8 | 255 | Y | Y | Y | PC3 (~900 bp) | LnCap = lymph node |
| | | GCCTTTGA | | | | | | Not in LnCap | cell line |
| | R | AGGTAGCCGAGA | 11 | | | | (~900 bp) | Both products were | PC3 = bone cell |
| | | CTGGAGCCTAG | | | | | | seen in all samples | line (androgen dependent) |
| | | | | | | | | | Results suggest CAIX |
| | | | | | | | | | is up-regulated in |
| | | | | | | | | | aggressive tumours |

Following RT-PCR, normal male samples show the correct (expected/calculated) size product, whilst alternative splicing is evident in cell line samples (LnCaP and PC3) and patient samples (NEOM, BPH, LocCaP and MetCaP).

EZH2 is a transcription repressor which modulates a cell growth pathway. Over expression of EZH2 leads to cancer development. The normal EZH2 product has 327 by and the alternatively spliced product observed in CaP has 390 bp. Both forms of EZH2 (normal and additional product) are observed in all patient groups and in cell lines. The demonstration of a larger splice form suggests mutation leading to an additional splice site within an intron, resulting in the inclusion of additional material.

e-cad is down-regulated in CaP especially in high Gleason score tumours and locally advanced and metastatic tumours. The normal e-cad has product 616 by and the alternatively spliced product has only 300 bp. The primers used for this RT-PCR are both located in exon 16. This suggests the possibility of base mutation within the exon, resulting in an additional splice recognition site and leading to the splicing out of exonic sequence. Alternatively spliced form was seen in both cell lines tested. Patient samples were not tested.

CAIX is regulated by HIF-1α, CAIX is induced during hypoxia and plays a role in the early establishment and development of cancer. qRT-PCR shows that CAIX is up-regulated in LocCaP and down-regulated in MetCaP. The normal CAIX product has 255 bp and the alternatively spliced product is 900 bp in length. The primers used to detect the alternatively spliced form span exons 8-11. The additional product includes parts of introns 9-10 and 10-11. The additional product was demonstrated in PC3 cell line (not in LnCaP cell line) and all patient samples. LnCaP is a lymph node cell line, whereas PC3 is a bone cell line and, therefore, androgen dependent. This would suggest that CAIX is up-regulated in aggressive tumours.

1. A method for determining the presence of prostate cancer in a subject which method comprises determining the level of expression of one or more markers in a blood sample from the subject, wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR.

2. A method for determining the stage of prostate cancer in a subject, which method comprises determining the level of expression of one or more markers in a blood sample from the subject, wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR.

3. A method according to claim 2, for discriminating between benign prostatic hyperplasia and malignant prostate cancer.

4. A method according to claim 2 for discriminating between localised invasive prostate cancer and metastatic prostate cancer.

5. A method for monitoring the response of a subject to prostate cancer treatment, which method comprises determining the level of expression of one or more markers in a blood sample from the subject, wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR.

6. A method for determining the aggressiveness of prostate cancer in a subject, which method comprises monitoring the level of expression of one or more markers in a blood sample from the subject, wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR.

7. A method according to claim 1 wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR.

8. A method according to claim 7, wherein said one or more markers comprises RECK and Clusterin, RECK and MTSP-1, Clusterin and MTSP-1 or RECK, Clusterin and MTSP-1.

9. A method according to claim 2, wherein said one or more markers comprise at least one of RECK, Clusterin, HIF-1α, E2F3, MMP9, e-cad and E2F4.

10. A method according to claim 2, wherein said one or more markers comprise at least one of RECK, Clusterin, HIF-1α, IGFBP-3, E2F4, caveolin, MMP9, PIM-1 and MTSP-1.

11. A method according to claim 5, wherein said one or more markers comprise at least one of RECK, Clusterin, MMP9, MTSP-1 and E2F3.

12. A method according to claim 6, wherein said one or more markers comprise at least one of E2F3 and CAIX.

13. A method according to claim 1, wherein the level of expression of one or more of the markers is determined by relative quantitative RT-PCR.

14. A method according to claim 13, wherein the relative quantification is calculated as a ratio of the amount of marker PCR product to the amount of a control PCR product.

15. A method according to claim 1, which method further comprises determining the level of expression of one or more of AMACR, PSA and FAS in a blood sample from the subject.

16. A method according to claim 1 which further comprises determining the presence or absence of one or more alternative splice variant of one or more marker.

17. A method according to claim 16, wherein said one or more alternative splice variant is selected from splice variants of EZH2, e-cad and CAIX.

18. A method according to claim 6, wherein the presence or absence of a CAXII alternative splice variant is determined.

19. A method according to claim 16, wherein the presence or absence of the one or more splice variants is determined by RT-PCR.

20. A method according to claim 1, wherein the subject has an enlarged prostate.

21. A test kit suitable for use in a method for determining the presence of prostate cancer in a subject, which test kit comprises means for determining the level of expression of one or more markers in a blood sample from the subject, wherein said one or more markers comprise at least one of E2F3, c-met, pRB, EZH2, e-cad, CAXII, CAIX, HIF-1α, Jagged, PIM-1, hepsin, RECK, Clusterin, MMP9, MTSP-1, MMP24, MMP15, IGFBP-2, IGFBP-3, E2F4, caveolin, EF-1A, Kallikrein 2, Kallikrein 3 and PSGR.

22. A test kit according to claim 21, which further comprises means for determining the level of expression of one or more of AMACR, PSA and FAS in a blood sample from the subject.

23. A test kit according to claim 21, which further comprises an internal control and means for determining the level of expression of the internal control.

24. A test kit according to claim 23, wherein the internal control is a gene encoding GAPDH, α-actin, β-actin or other enzyme of the glycolytic pathway.

26. A method for the treatment of prostate cancer in a subject, which method comprises:
(a) determining whether the subject has prostate cancer by use of a method according to claim 1; and
(b) administering to a subject identified in (a) as having prostate cancer, a therapeutically effective amount of an agent used in the treatment of prostate cancer.


SEQUENCE LISTING (60 sequences)

SEQ ID NO: 1 - DNA, 20 bp, homo sapiens
aatatggcgtagtatctccg

SEQ ID NO: 2 - DNA, 20 bp, homo sapiens
cttcccaaacatacacccac

SEQ ID NO: 3 - DNA, 16 bp, homo sapiens
aatttccgaggtgggc

SEQ ID NO: 4 - DNA, 18 bp, homo sapiens
gaaagtacacggggatag

SEQ ID NO: 5 - DNA, 20 bp, homo sapiens
gcagtgcagcatgtagtgat

SEQ ID NO: 6 - DNA, 20 bp, homo sapiens
caggagcgagaggacattgg

SEQ ID NO: 7 - DNA, 20 bp, homo sapiens
tttctgctcctgcacacact

SEQ ID NO: 8 - DNA, 21 bp, homo sapiens
aggtgctgctgaggttggaga

SEQ ID NO: 9 - DNA, 22 bp, homo sapiens
cgtatgccccgctgaatctcgt

SEQ ID NO: 10 - DNA, 22 bp, homo sapiens
tggccaatcatccgtgctcatc

SEQ ID NO: 11 - DNA, 20 bp, homo sapiens
cgcatcttgataaggcctct

SEQ ID NO: 12 - DNA, 20 bp, homo sapiens
taccttccatgttgcagact

SEQ ID NO: 13 - DNA, 20 bp, homo sapiens
ccgagcgacgcagcctttga

SEQ ID NO: 14 - DNA, 23 bp, homo sapiens
aggtagccgagactggagcctag

SEQ ID NO: 15 - DNA, 27 bp, homo sapiens
ggacaaatggggacaggaaggatcaag

SEQ ID NO: 16 - DNA, 27 bp, homo sapiens
gaggacatttcatgctgtcaaaatgag

SEQ ID NO: 17 - DNA, 19 bp, homo sapiens
tgtcccgatggcgagtgtt

SEQ ID NO: 18 - DNA, 20 bp, homo sapiens
cctgttggccatagtactgc

SEQ ID NO: 19 - DNA, 19 bp, homo sapiens
atcaggggccaggttttct

SEQ ID NO: 20 - DNA, 20 bp, homo sapiens
aaaggctgctatttgctggg

SEQ ID NO: 21 - DNA, 25 bp, homo sapiens
gccacctgtgtgcttattggtatcc

SEQ ID NO: 22 - DNA, 28 bp, homo sapiens
gacacaataggagtgcgagaggacattg

SEQ ID NO: 23 - DNA, 21 bp, homo sapiens
cctatacgttgcttgtggagg

SEQ ID NO: 24 - DNA, 20 bp, homo sapiens
tgccagggctcattacagat

SEQ ID NO: 25 - DNA, 24 bp, homo sapiens
tcgccattctctctttcctgcaca

SEQ ID NO: 26 - DNA, 24 bp, homo sapiens
tggaatagacacggctgatgcact

SEQ ID NO: 27 - DNA, 24 bp, homo sapiens
aagaagctggctgacatgtacgga

SEQ ID NO: 28 - DNA, 24 bp, homo sapiens
aaccaccagcaacgtgatttctgc

SEQ ID NO: 29 - DNA, 20 bp, homo sapiens
taccaccctggggttatgaa

SEQ ID NO: 30 - DNA, 20 bp, homo sapiens
tagtaacaagacggtggggc

SEQ ID NO: 31 - DNA, 20 bp, homo sapiens
ccagacactcacagcaagga

SEQ ID NO: 32 - DNA, 20 bp, homo sapiens
gtcctccagacaaccctcag

SEQ ID NO: 33 - DNA, 20 bp, homo sapiens
catgctggagccaagtgcta

SEQ ID NO: 34 - DNA, 22 bp, homo sapiens
gccaacaggaacagtaccaata

SEQ ID NO: 35 - DNA, 20 bp, homo sapiens
actgctggctgccttagaac

SEQ ID NO: 36 - DNA, 20 bp, homo sapiens
tgaacaggggaaccatcact

SEQ ID NO: 37 - DNA, 20 bp, homo sapiens
agtccaggaatgggtgtgag

SEQ ID NO: 38 - DNA, 20 bp, homo sapiens
ccaccacttcagctgtacga

SEQ ID NO: 39 - DNA, 19 bp, homo sapiens
agttcccggagtgagttga

SEQ ID NO: 40 - DNA, 20 bp, homo sapiens
cacctccactcctccctttc

SEQ ID NO: 41 - DNA, 20 bp, homo sapiens
aactggctgcggctttatgg

SEQ ID NO: 42 - DNA, 21 bp, homo sapiens
aggtcagatggtggttgttcc

SEQ ID NO: 43 - DNA, 20 bp, homo sapiens
caccaagttcgtgtcccttc

SEQ ID NO: 44 - DNA, 20 bp, homo sapiens
gcccgataccttccaaaaca

SEQ ID NO: 45 - DNA, 19 bp, homo sapiens
ctaccacaaggagtcggct

SEQ ID NO: 46 - DNA, 20 bp, homo sapiens
tgtcctgggtcctctgtact

SEQ ID NO: 47 - DNA, 20 bp, homo sapiens
ggataaccaaatgtgccgtg

SEQ ID NO: 48 - DNA, 20 bp, homo sapiens
caatagccagttcacagcag

SEQ ID NO: 49 - DNA, 21 bp, homo sapiens
tatgaaggagctggccgtgtt

SEQ ID NO: 50 - DNA, 20 bp, homo sapiens
caggccatgcttgtcacagt

SEQ ID NO: 51 - DNA, 21 bp, homo sapiens
tgccgtagagaaatggaagac

SEQ ID NO: 52 - DNA, 20 bp, homo sapiens
tagcagtgcacgtcctcctt

SEQ ID NO: 53 - DNA, 20 bp, homo sapiens
tccaggacaggttcttcacc

SEQ ID NO: 54 - DNA, 20 bp, homo sapiens
tgctgagcctcgtgtatcat

SEQ ID NO: 55 - DNA, 16 bp, homo sapiens
tacctggctgtccgag

SEQ ID NO: 56 - DNA, 16 bp, homo sapiens
gatgcaacccgcaagg

SEQ ID NO: 57 - DNA, 17 bp, homo sapiens
actacttgaacgaatgg

SEQ ID NO: 58 - DNA, 16 bp, homo sapiens
ttagtcatgcgtagtg

SEQ ID NO: 59 - DNA, 20 bp, homo sapiens
ccgagcgacgcagcctttga

SEQ ID NO: 60 - DNA, 23 bp, homo sapiens
aggtagccgagactggagcctag